SEQUENCE LISTING (1 sequence)

SEQ ID NO: 1 - DNA, 30 bp, Artificial Sequence
  Description of Artificial Sequence Synthetic oligonucleotide
tcgaacgttcgaacgttcgaacgttcgaat

(19) United States
(12) Patent Application Publication
(10) Pub. No.: US 2024/0180952 A1
(43) Pub. Date: Jun. 6, 2024

(54) CANCER THERAPY USING TOLL-LIKE RECEPTOR AGONISTS

(71) Applicant: TriSalus Life Sciences, Inc., Westminster, CO (US)

(72) Inventors: Steven C. Katz, East Greenwich, RI (US); Bryan F. Cox, Muscatine, IA (US); David Benjamin Jaroch, Arvada, CO (US)

(73) Assignee: TriSalus Life Sciences, Inc., Westminster, CO (US)

(21) Appl. No.: 18/285,063

(22) PCT Filed: Mar. 31, 2022
(86) PCT No.: PCT/US2022/022801

§ 371 Date: Sep. 29, 2023

Related U.S. Application Data
(60) Provisional application No. 63/298,589, filed on Jan. 11, 2022.

(51) Int. Cl. A61K 31/711; A61K 9/00; A61K 39/00; C07K 16/28

(52) U.S. Cl. CPC A61K 31/711; A61K 9/0019; C07K 16/2818; A61K 2039/505

(57) ABSTRACT

Embodiments of the present invention provide for methods of treating cancer and methods of delivering toll-like receptor (TLR) agonists to solid tumors in the liver using a locoregional therapy through the vasculature. In one aspect, the present invention relates to a method of treating primary liver cancers such as hepatocellular carcinoma (HCC) and intra-hepatic cholangiocarcinoma (ICC) comprising administering TLR9 agonists to the liver.

### CROSS-REFERENCE TO RELATED APPLICATION(S)

This application claims the benefit of U.S. Provisional Patent Application No. 63/169,674, which was filed on Apr. 1, 2021 and U.S. Provisional Patent Application No. 63/298,589, which was filed on Jan. 11, 2022, both of which are incorporated by reference in their entireties.

### SEQUENCE LISTING

The instant application contains a Sequence Listing which has been submitted electronically in ASCII format and is hereby incorporated by reference in its entirety. Said ASCII copy, created on Sep. 16, 2020, is named A372-502_SL.txt and is 484 bytes in size.

### FIELD OF THE INVENTION

The present disclosure relates generally to methods of treating cancer and methods of delivering toll-like receptor (TLR) agonists to solid tumors in the liver using a locoregional therapy through the vasculature.

### BACKGROUND OF THE INVENTION

Cancer is a devastating disease that involves the unchecked growth of cells, which may result in the growth of solid tumors in a variety of organs such as the skin, liver, and pancreas. Tumors may first present in any number of organs or may be the result of metastases or spread from other locations.

Hepatocellular carcinoma (HCC) and intrahepatic cholangiocarcinoma (ICC) are the most common primary liver tumors and are among the solid organ malignancies in which progression within the liver results in a rapidly progressive and often lethal condition.

HCC represents more than 90% of cases of primary liver tumors. Both HCC and ICC patients share many of the same features and challenges with respect to treatment. For both diseases, the 5-year survival rate is <20%.

Surgery has been the only potential curative treatment for primary liver cancers such as HCC and ICC; however, approximately 70% of patients are diagnosed at advanced stages due to an absence of specific symptoms, and even following potentially curative resection, tumor relapse is frequent. Given their effectiveness in other malignancies, checkpoint inhibitors (CPI) have also been tried in patients with HCC and ICC; however, their success to date has been limited due to drug delivery challenges and suppression of immune responses in the liver.

Accordingly, there remains a need for a safe and effective therapy for the treatment of primary liver cancers such as HCC and ICC.

### SUMMARY OF THE INVENTION

The present invention relates to methods of treating primary liver cancers using a therapeutically effective amount of a toll-like receptor (TLR) agonist.

In one aspect, the present invention relates to a method of treating primary liver cancers comprising administering a TLR agonist through a device by hepatic arterial infusion (HAI). According to another embodiment, the treatment of primary liver cancers comprises administering a TLR agonist through a device by portal vein infusion (PVI).

In some embodiments, the TLR agonist is administered through pressure-enabled drug delivery (PEDD), which includes the administration of a therapeutic through a device, such as a catheter device. In some embodiments, the catheter device comprises a one-way valve that responds dynamically to local pressure changes. In some embodiments, the catheter device generates, causes, and/or contributes to a net increase in fluid pressure within the vessel and/or target tissue or tumor. In some embodiments, the catheter device generates, causes, and/or contributes to a net decrease in fluid pressure within the vessel and/or target tissue or tumor. In some embodiments, the catheter device generates, causes, and/or contributes to first a decrease, then an increase in fluid pressure within the vessel and/or target tissue or tumor.

In some embodiments, the TLR agonist is administered through a pressure-enabled device, such as one that modulates vascular pressure.

In some embodiments, the amount of TLR agonist administered is in the range of about 0.01-20 mg, or at least one of 0.5 mg, 2 mg, 4 mg, or 8 mg.

In some embodiments, the TLR agonist is administered in a solution in the range of 1-100 mL, or at least one of 10 mL, 25 mL, or 50 mL.

In some embodiments, the TLR agonist is administered in the range of 0.0001-20 mg/mL, or at least one of 0.01 mg/mL, 0.04 mg/mL, 0.08 mg/mL, or 0.16 mg/mL.

In some embodiments, the TLR agonist is administered for a period of time of about 10-200 minutes. In another embodiment, the TLR agonist is administered for a period of time of about 10-60 minutes. In another embodiment, the TLR agonist is administered for a period of time of about 25 minutes.

In some embodiments, the TLR agonist is administered in combination with one or more CPIs. In some embodiments, the CPIs are administered systemically, either concurrently, before, or after the administration of the TLR agonist. In some embodiments, the CPIs include at least one of nivolumab, pembrolizumab, and ipilimumab.

In some embodiments, administration of the TLR agonist comprises a dosing regimen comprising cycles. In some embodiments, one or more of the cycles comprise the administration of the TLR agonist via a catheter device by HAI followed by the systemic administration of one or more CPIs.

In some embodiments, one cycle of the dosing regimen comprises the administration of the TLR agonist once per week over three consecutive weeks. In some embodiments, the dosing regimen comprises two cycles.

In some embodiments, the administration of a TLR agonist through an intravascular device to the liver results in a reduction of myeloid-derived suppressor cells (MDSC) or the functional alteration of MDSCs to limit immunosuppression. In some embodiments, the administration of a TLR agonist through an intravascular device to the liver results in antitumor effects.

In some embodiments, the TLR agonist is a TLR9 agonist. In some embodiments, the TLR9 agonist is SD-101.

These and other objects, features, and advantages of the exemplary embodiments of the present disclosure will become apparent upon reading the following detailed description of the exemplary embodiments of the present disclosure, when taken in conjunction with the entire specification.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure.

FIG. 1 illustrates the structure of SD-101.

FIG. 2 illustrates an in vitro analysis of human PMBC harvested from healthy donors and treated with IL6 and GM-CSF to induce MDSC.

FIG. 3 illustrates an in vitro analysis of murine bone marrow cells, in which GM-CSF was used to induce MDSC, from mice with or without tumor.

FIG. 4 illustrates cross-section images of swine liver lobes after the infusion of SD-101.

FIG. 5 illustrates the overall study design of a phase 1b/2 pressure enabled regional immune-oncology study of HAI of SD-101 with systemic checkpoint blockade for HCC and ICC.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended paragraphs.

### DETAILED DESCRIPTION

The following description of embodiments provides non-limiting representative examples referencing numerals to particularly describe features and teachings of different aspects of the invention. The embodiments described should be recognized as capable of implementation separately, or in combination, with other embodiments from the description of the embodiments. A person of ordinary skill in the art reviewing the description of embodiments should be able to understand the different described aspects of the invention. The description of embodiments should facilitate understanding of the invention to such an extent that other implementations, not specifically covered but within the knowledge of a person of skill in the art having read the description of embodiments, would be understood to be consistent with application of the invention.

### Toll-Like Receptor Agonists

Toll-like receptors are pattern recognition receptors that can detect microbial pathogen-associated molecular patterns (PAMPs). TLR stimulation, such as TLR9 stimulation, can not only provide broad innate immune stimulation, but can also specifically address the dominant drivers of immunosuppression in the liver. TLR1-10 are expressed in humans and recognize a diverse variety of microbial PAMPs. In this regard, TLR9 can respond to unmethylated CpG-DNA, including microbial DNA. CpG refers to the motif of a cytosine and guanine dinucleotide 1. TLR9 is constitutively expressed in B cells, plasmacytoid dendritic cells (pDCs), activated neutrophils, monocytes/macrophages, T cells, and MDSCs. TLR9 is also expressed in non-immune cells, including keratinocytes and gut, cervical, and respiratory epithelial cells. TLR9 can bind to its agonists within endosomes. Signaling may be carried out through MYD88/IkB/NfκB to induce pro-inflammatory cytokine gene expression. A parallel signaling pathway through IRF7 induces type 1 and 2 interferons (e.g. IFN-α, IFN-γ, etc.) which stimulate adaptive immune responses. Further, TLR9 agonists can induce cytokine and IFN production and functional maturation of antigen presenting dendritic cells.

According to an embodiment, a TLR9 agonist can reduce and reprogram MDSCs. MDSCs are key drivers of immunosuppression in the liver. MDSCs also drive expansion of other suppressor cell types such as T regulatory cells (Tregs), tumor-associated macrophages (TAMs), and cancer-associated fibroblasts (CAFs). MDSCs may downregulate immune cells and interfere with the effectiveness of immunotherapeutics. Further, high MDSC levels generally predict poor outcomes in cancer patients. In this regard, reducing, altering, or eliminating MDSCs is thought to improve the ability of the host's immune system to attack the cancer as well as the ability of the immunotherapy to induce more beneficial therapeutic responses. In an embodiment, TLR9 agonists may convert MDSCs into immunostimulatory M1 macrophages, convert immature dendritic cells to mature dendritic cells, and expand effector T cells to create a responsive tumor microenvironment to promote anti-tumor activity.

According to an embodiment, synthetic CpG-oligonucleotides (CPG-ONs) mimicking the immunostimulatory nature of microbial CpG-DNA can be developed for therapeutic use. According to an embodiment, the oligonucleotide is an oligodeoxynucleotide (ODN). There are a number of different CpG-ODN class types, e.g. Class A, Class B, Class C, Class P, and Class S, which share certain structural and functional features. In this regard, Class A type CPG-ODNs (or CPG-A ODNs) are associated with pDC maturation with little effect on B cells as well as the highest degree of IFNα induction; Class B type CPG-ODNs (or CPG-B ODNs) strongly induce B-cell proliferation, activate pDC and monocyte maturation, NK cell activation, and inflammatory cytokine production; and Class C type CPG-ODNs (or CPG-C ODNs) can induce B-cell proliferation and IFN-α production.

Further, according to an embodiment, CPG-C ODNs can be associated with the following attributes: (i) unmethylated dinucleotide CpG motifs, (ii) juxtaposed CpG motifs with flanking nucleotides (e.g. AACGTTCGAA), (iii) a complete phosphorothioate (PS) backbone that links the nucleotides (as opposed to the natural phosphodiester (PO) backbones found in bacterial DNA), and (iv) a self-complimentary, palindromic sequence (e.g. AACGTT). In this regard, CPG-C ODNs may bind themselves due to their palindromic nature, thereby producing double-stranded duplex or hairpin structures.

Further, according to an embodiment, the CPG-C ODNs can include one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In an embodiment, the CpG-C ODNs are 12 to 100 bases in length, preferably 12 to 50 bases in length, preferably 12 to 40 bases in length, or preferably 12-30 bases in length. In an embodiment, the CpG-C ODN is 30 bases in length. In an embodiment, the ODN is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In an embodiment, the ODN is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length.

In an embodiment, the at least one palindromic sequence is 8 to 97 bases in length, preferably 8 to 50 bases in length, or preferably 8 to 32 bases in length. In an embodiment, the at least one palindromic sequence is at least (lower limit) 8, 10, 12, 14, 16, 18, 20, 22, 24, 26, 28, or 30 bases in length. In an embodiment, the at least one palindromic sequence is at most (upper limit) 50, 48, 46, 44, 42, 40, 38, 36, 34, 32, 30, 28, 26, 24, 22, 20, 18, 16, 14, 12 or 10 bases in length.

In an embodiment, the CpG-C ODN can comprise the sequence of SEQ ID NO: 1.

According to an embodiment, the CpG-C ODN can comprise the SD-101. SD-101 is a 30-mer phosphorothioate oligodeoxynucleotide, having the following sequence: 5′-TCG AAC GTT CGA ACG TTC GAA CGT TCG AAT-3′ (SEQ ID NO: 1)

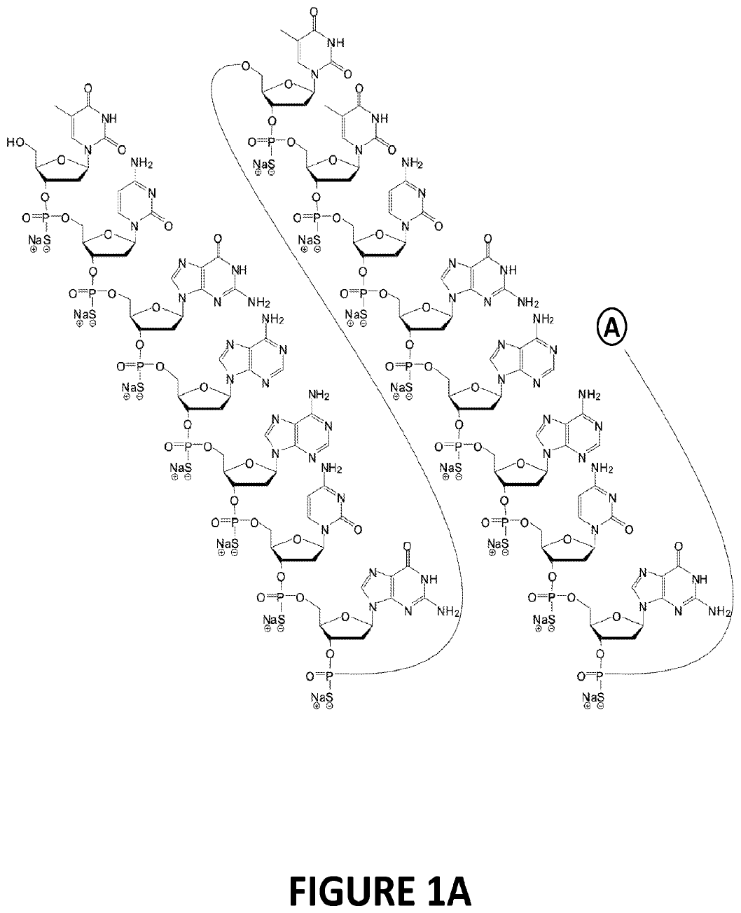

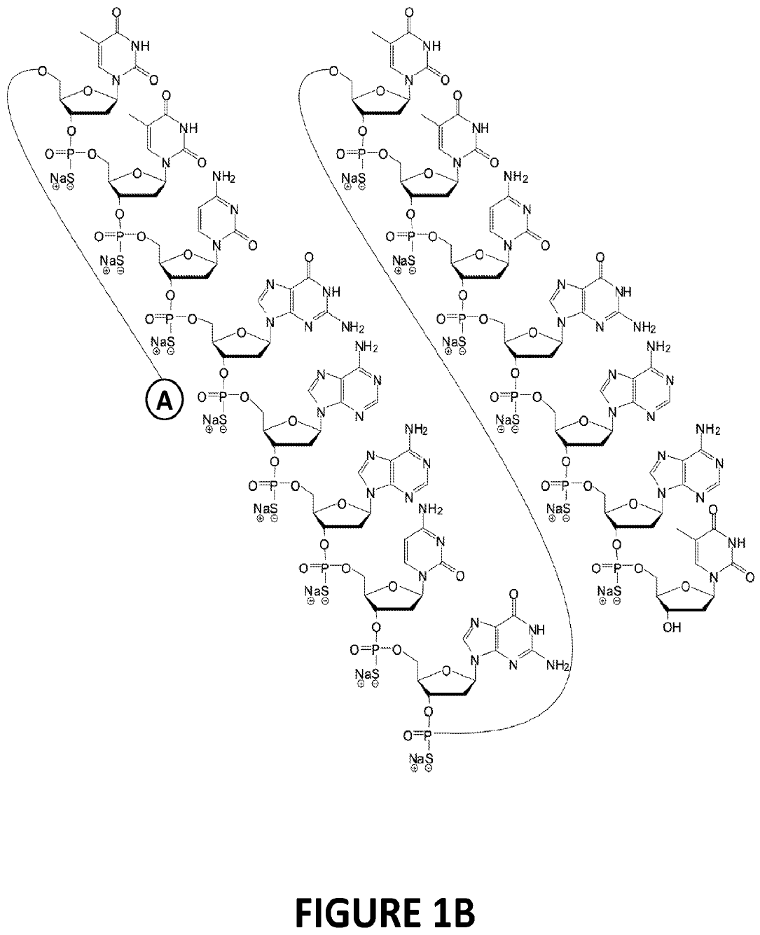

SD-101 drug substance is isolated as the sodium salt. The structure of SD-101 is illustrated in FIG. 1.

The molecular formula of SD-101 free acid is C₂₉₃ H₃₆₉ N₁₁₂ O₁₄₉ P₂₉ S₂₉ and the molecular mass of the SD-101 free acid is 9672 Daltons. The molecular formula of SD-101 sodium salt is C₂₉₃ H₃₄₀ N₁₁₂ O₁₄₉ P₂₉ S₂₉ Na₂₉ and the molecular mass of the SD-101 sodium salt is 10,309 Daltons.

Further, according to an embodiment, the CPG-C ODN sequence can correspond to SEQ ID NO: 172 as described in U.S. Pat. No. 9,422,564, which is incorporated by reference herein in its entirety.

In an embodiment, the CpG-C ODN can comprise a sequence that has at least 75% homology to any of the foregoing, such as SEQ ID NO:1.

According to another embodiment the CPG-C ODN sequence can correspond to any one of the other sequences described in U.S. Pat. No. 9,422,564. Further, the CPG-C ODN sequence can also correspond to any of the sequences described in U.S. Pat. No. 8,372,413, which is also incorporated by reference herein in its entirety.

According to an embodiment, any of the CPG-C ODNs discussed herein may be present in their pharmaceutically acceptable salt forms. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In an embodiment, the CpG-C ODNs are in the ammonium, sodium, lithium, or potassium salt forms. In one preferred embodiment, the CpG-C ODNs are in the sodium salt form. The CpG-C ODN may be provided in a pharmaceutical solution comprising a pharmaceutically acceptable excipient. Alternatively, the CpG-C ODN may be provided as a lyophilized solid, which is subsequently reconstituted in sterile water, saline or a pharmaceutically acceptable buffer before administration. Pharmaceutically acceptable excipients of the present disclosure include for instance, solvents, bulking agents, buffering agents, tonicity adjusting agents, and preservatives. In an embodiment, the pharmaceutical compositions may comprise an excipient that functions as one or more of a solvent, a bulking agent, a buffering agent, and a tonicity adjusting agent (e.g. sodium chloride in saline may serve as both an aqueous vehicle and a tonicity adjusting agent). The pharmaceutical compositions of the present disclosure are suitable for parenteral and/or percutaneous administration.

In an embodiment, the pharmaceutical compositions comprise an aqueous vehicle as a solvent. Suitable vehicles include for instance sterile water, saline solution, phosphate buffered saline, and Ringer's solution. In an embodiment, the composition is isotonic.

The pharmaceutical compositions may comprise a bulking agent. Bulking agents are particularly useful when the pharmaceutical composition is to be lyophilized before administration. In an embodiment, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In an embodiment, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin, and mannitol.

The pharmaceutical compositions may comprise a preservative. Suitable preservatives include for instance antioxidants and antimicrobial agents. However, in an embodiment, the pharmaceutical composition is prepared under sterile conditions and is in a single use container, and thus does not necessitate inclusion of a preservative.

Table 1 describes the batch formula for SD-101 Drug Product—16 g/L:

TABLE 1

| | | | | Clinical Lot | |
|---|---|---|---|---|---|
| | | | Amount per | DVXA05 | |
| Ingredient | Grade | Concentration | batch | Batch Size 2.224 L | |
| SD-101 Drug | GMP | 1.6% | 16.00 g | 35.584 | g¹ |
| Substance* | | | | | |
| Sodium | USP/NF, EP | 1.02% | 1.02 g | 2.268 | g |
| phosphate, | | | | | |
| dibasic | | | | | |
| anhydrous | | | | | |
| Sodium | USP | 0.34% | 0.34 g | 0.747 | g |
| phosphate, | | | | | |
| monobasic | | | | | |
| anhydrous | | | | | |
| Sodium | USP/NF, EP | 7.31% | 7.31 g | 16.257 | g |

| | | | | |
|---|---|---|---|---|
| chloride | | | | |
| Sterile Water | USP/NF, EP | QS | QS | 2.224 L (kg) (QS) |
| for Injection | | | | |

¹Quantity based upon measured content in solution (to exclude moisture present in lyophilized powder)
*SD-101 Drug Substance in Table 1 reflects the totality of all oligonucleotide content, including SD-101.

In some embodiments, the unit dose strength may include from about 0.1 mg/mL to about 20 mg/mL. In one embodiment, the unit dose strength of SD-101 is 13.4 mg/mL.

In some embodiments, the amount of SD-101 administered is in the range of about 0.01-20 mg, or at least one of 0.5 mg, 2 mg, 4 mg, or 8 mg.

In some embodiments, SD-101 is administered in a solution in the range of 1-100 mL, or at least one of 10 mL, 25 mL, or 50 mL.

In some embodiments, an administered dose of SD-101 is in the range of 0.0001-20 mg/mL. In some embodiments, the administered dose of SD-101 is one of 0.01 mg/mL, 0.04 mg/mL, 0.08 mg/mL, or 0.16 mg/mL.

CpG-C ODNs may contain modifications. Suitable modifications can include but are not limited to, modifications of the 3′OH or 5′OH group, modifications of the nucleotide base, modifications of the sugar component, and modifications of the phosphate group. Modified bases may be included in the palindromic sequence as long as the modified base(s) maintains the same specificity for its natural complement through Watson-Crick base pairing (e.g. the palindromic portion of the CpG-C ODN remains self-complementary). Examples of modifications of the 5′OH group can include biotin, cyanine 5.5, the cyanine family of dyes, Alexa Fluor 660, the Alexa Fluor family of dyes, IRDye 700, IRDye 800, IRDye 800CW, and the IRDye family of dyes.

CpG-C ODNs may be linear, may be circular or include circular portions and/or a hairpin loop. CpG-C ODNs may be single stranded or double stranded. CpG-C ODNs may be DNA, RNA or a DNA/RNA hybrid.

CpG-C ODNs may contain naturally-occurring or modified, non-naturally occurring bases, and may contain modified sugar, phosphate, and/or termini. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. In an embodiment, CpG-C ODNs have only phosphorothioate linkages, only phosphodiester linkages, or a combination of phosphodiester and phosphorothioate linkages.

Sugar modifications known in the field, such as 2′-alkoxy-RNA analogs, 2′-amino-RNA analogs, 2′-fluoro-DNA, and 2′-alkoxy- or amino-RNA/DNA chimeras and others described herein, may also be made and combined with any phosphate modification. Examples of base modifications include but are not limited to addition of an electron-withdrawing moiety to C-5 and/or C-6 of a cytosine of the CpG-C ODN (e.g. 5-bromocytosine, 5-chlorocytosine, 5-fluorocytosine, 5-iodocytosine) and C-5 and/or C-6 of a uracil of the CpG-C ODN (e.g. 5-bromouracil, 5-chlorouracil, 5-fluorouracil, 5-iodouracil). As noted above, use of a base modification in a palindromic sequence of a CpG-C ODN should not interfere with the self-complementarity of the bases involved for Watson-Crick base pairing. However, outside of a palindromic sequence, modified bases may be used without this restriction. For instance, 2′-O-methyl-uridine and 2′-O-methyl-cytidine may be used outside of the palindromic sequence, whereas, 5-bromo-2′-deoxycytidine may be used both inside and outside the palindromic sequence. Other modified nucleotides, which may be employed both inside and outside of the palindromic sequence include 7-deaza-8-aza-dG, 2-amino-dA, and 2-thio-dT.

Duplex (i.e. double stranded) and hairpin forms of most ODNs are often in dynamic equilibrium, with the hairpin form generally favored at low oligonucleotide concentration and higher temperatures. Covalent interstrand or intrastrand cross-links increase duplex or hairpin stability, respectively, towards thermal-, ionic-, pH-, and concentration-induced conformational changes. Chemical cross-links can be used to lock the polynucleotide into either the duplex or the hairpin form for physicochemical and biological characterization. Cross-linked ODNs that are conformationally homogeneous and are “locked” in their most active form (either duplex or hairpin form) could potentially be more active than their uncross-linked counterparts. Accordingly, some CpG-C ODNs of the present disclosure can contain covalent interstrand and/or intrastrand cross-links.

The techniques for making polynucleotides and modified polynucleotides are known in the art. Naturally occurring DNA or RNA, containing phosphodiester linkages, may be generally synthesized by sequentially coupling the appropriate nucleoside phosphoramidite to the 5′-hydroxy group of the growing ODN attached to a solid support at the 3′-end, followed by oxidation of the intermediate phosphite triester to a phosphate triester. Using this method, once the desired polynucleotide sequence has been synthesized, the polynucleotide is removed from the support, the phosphate triester groups are deprotected to phosphate diesters and the nucleoside bases are deprotected using aqueous ammonia or other bases.

The CpG-C ODN may contain phosphate-modified oligonucleotides, some of which are known to stabilize the ODN. Accordingly, some embodiments include stabilized CpG-C ODNs. The phosphorous derivative (or modified phosphate group), which can be attached to the sugar or sugar analog moiety in the ODN, can be a monophosphate, diphosphate, triphosphate, alkylphosphonate, phosphorothioate, phosphorodithioate, phosphoramidate or the like.

CpG-C ODNs can comprise one or more ribonucleotides (containing ribose as the only or principal sugar component), deoxyribonucleotides (containing deoxyribose as the principal sugar component), modified sugars or sugar analogs. Thus, in addition to ribose and deoxyribose, the sugar moiety can be pentose, deoxypentose, hexose, deoxyhexose, glucose, arabinose, xylose, lyxose, and a sugar analog cyclopentyl group. The sugar can be in pyranosyl or in a furanosyl form. In the CpG-C oligonucleotide, the sugar moiety is preferably the furanoside of ribose, deoxyribose, arabinose or 2′-O-alkylribose, and the sugar can be attached to the respective heterocyclic bases in either anomeric configuration. The preparation of these sugars or sugar analogs and the respective nucleosides wherein such sugars or analogs are attached to a heterocyclic base (nucleic acid base) per se is known, and therefore need not be described here. Sugar modifications may also be made and combined with any phosphate modification in the preparation of a CpG-C ODN.

The heterocyclic bases, or nucleic acid bases, which are incorporated in the CpG-C ODN can be the naturally-occurring principal purine and pyrimidine bases, (namely uracil, thymine, cytosine, adenine and guanine, as mentioned above), as well as naturally-occurring and synthetic modifications of said principal bases. Thus, a CpG-C ODN may include one or more of inosine, 2′-deoxyuridine, and 2-amino-2′-deoxyadenosine.

According to another embodiment, the CPG-ODN is one of a Class A type CPG-ODNs (CPG-A ODNs), a Class B type CPG-ODNs (CPG-B ODNs), a Class P type CPG-ODNs (CPG-P ODN), and a Class S type CPG-ODNs (CPG-S ODN). In this regard, the CPG-A ODN can be CMP-001.

In another embodiment, the CPG-ODN can be tilsotolimod (IMO-2125).

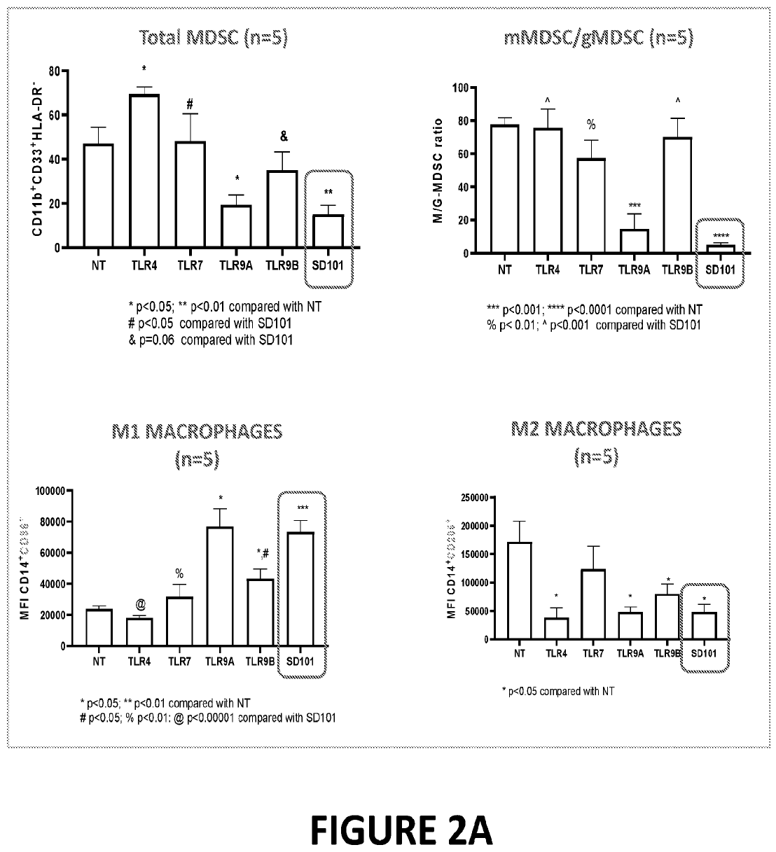

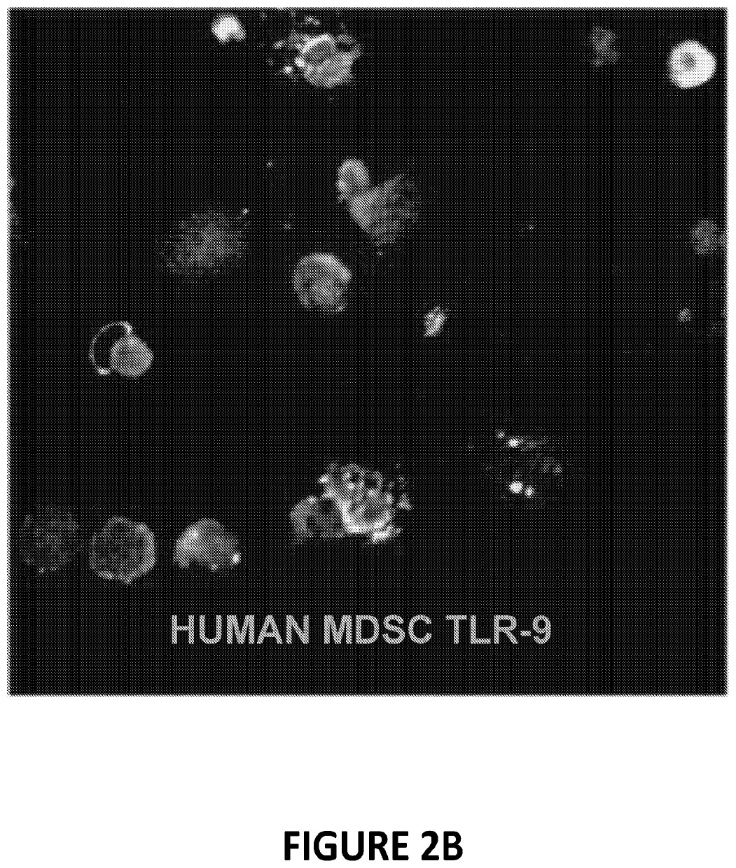

Further, according to an embodiment, in vitro studies of human peripheral blood mononuclear cells (PMBC) demonstrate that SD-101 is superior to Class B TLR9 agonist and a TLR7 agonist with respect to reduction of MDSC, reduction of M2 macrophages, and promotion of M1 macrophages. For example, FIG. 2 depicts an in vitro analysis of human PMBC harvested from healthy donors and treated with IL6 and GM-CSF to induce MDSC. In this regard, SD-101 demonstrated (i) better elimination of the dominant MDSC subset in the liver, e.g., M-MDSC and (ii) a more favorable effect on the M1/M2 macrophage ratio (i.e., induction of M1 and reduction of M2).

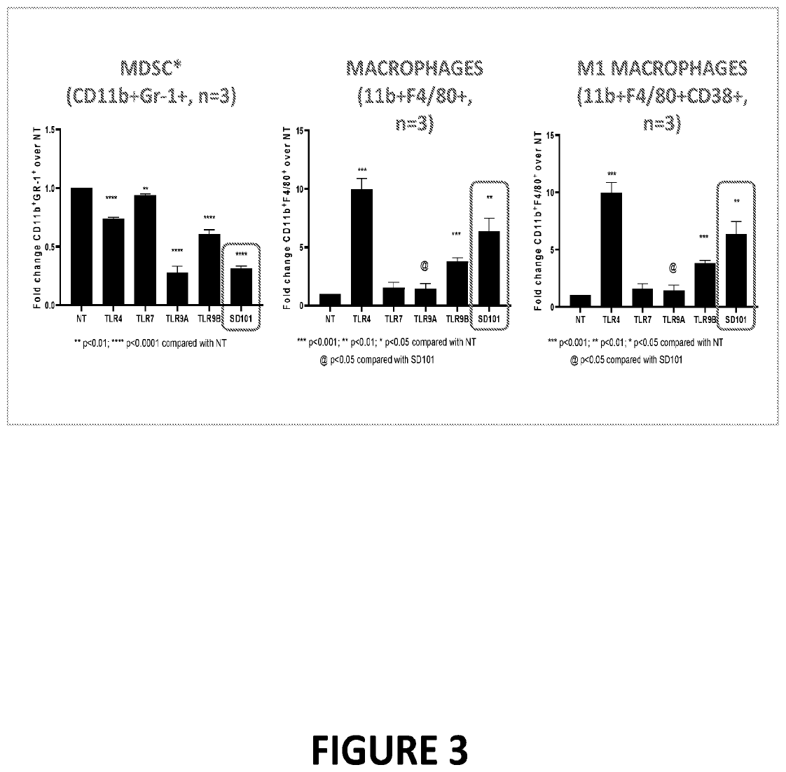

Further, according to an embodiment, in vitro studies of murine bone marrow cells demonstrate that SD-101 is similarly superior to a Class B TLR9 agonist and a TLR7 agonist for murine myeloid cell programming. For example, FIG. 3 depicts an in vitro analysis of murine bone marrow cells, in which GM-CSF was used to induce MDSC, from mice with or without tumor. In this regard, SD-101 was superior to class A TLR9, class B TLR9, and TLR7 agonists in reduction of MDSC and M2 macrophages, in addition to induction of M1 macrophages.

### Checkpoint Inhibitors

According to an embodiment, the TLR agonists of the present invention may be used in combination with one or more CPIs. The CPI can include a Programmed Death 1 receptor (PD-1) antagonist. A PD-1 antagonist can be any chemical compound or biological molecule that blocks binding of Programmed Cell Death 1 Ligand 1 (PD-L1) expressed on a cancer cell to PD-1 expressed on an immune cell (T cell, B cell or NKT cell) and preferably also blocks binding of PD-L2 Programmed Cell Death 1 Ligand 2 (PD-L2) expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and uses of the present invention in which a human individual is being treated, the PD-1 antagonist blocks binding of human PD-L1 to human PD-1, and preferably blocks binding of both human PD-L1 and PD-L2 to human PD-1.

According to an embodiment, the PD-1 antagonist can include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

According to an embodiment, the PD-1 antagonist can include an immunoadhesin that specifically binds to PD-1 or PD-L1, and preferably specifically binds to human PD-1 or human PD-L1, e.g. a fusion protein containing the extracellular or PD-1 binding portion of PD-L1 or PD-L2 fused to a constant region such as an Fc region of an immunoglobulin molecule.

According to an embodiment, the PD-1 antagonist can block PD-L1 expressed by tumor cells and MDSC, and other suppressive immune cells.

According to an embodiment, the PD-1 antagonist can inhibit the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain.

According to an embodiment, the PD-1 antagonist can be one of nivolumab, pembrolizumab, cemiplimab, and dostarlimab.

According to an embodiment, nivolumab is administered intravenously (IV) via a peripheral vein at a dose of 480 mg every four weeks (“Q4W”) or 240 mg every two weeks (“Q2W”). According to another embodiment, nivolumab is administered intravenously (IV) via a peripheral vein at a dose of nivolumab 360 mg every three weeks (“Q3W”). In another embodiment, nivolumab dosing is weight-based, at nivolumab 3 mg/kg Q2W or 10 mg/kg Q2W. In another embodiment, nivolumab dosing is weight-based at nivolumab 1 mg/kg Q3W. In yet another embodiment, nivolumab is administered concomitantly, at the same time, at about the same time, or on the same day with SD-101. In another embodiment, nivolumab is administered one a weekly, every other week, every three weeks, every four weeks, or on a monthly basis following the administration of one or more cycles of SD-101.

According to another embodiment, pembrolizumab is administered intravenously (IV) via a peripheral vein at a dose of 200 mg Q3W or 400 mg every 6 weeks (“Q6W”). In another embodiment, pembrolizumab is administered concomitantly, at the same time, at about the same time, or on the same day with SD-101.

According to another embodiment, the CPI can include a PD-L1 antagonist. In this regard, the PD-L1 antagonist can be one of atezolizumab, avelumab, and durvalumab.

According to another embodiment, the CPI can include a CTLA-4 antagonist. In this regard, the CTLA-4 antagonist can be ipilimumab.

According to an embodiment, ipilimumab is administered intravenously (IV) via a peripheral vein at a dose of 3 mg/kg every three weeks. In yet another embodiment, ipilimumab is administered concomitantly, at the same time, at about the same time, or on the same day with SD-101 and/or nivolumab. In another embodiment, ipilimumab is administered once a week, every other week, every three weeks, every four weeks, or on a monthly basis following the administration of one or more cycles of SD-101 and/or nivolumab.

### Devices to Achieve Locoregional Delivery

According to an embodiment, any of the above-described devices may comprise any device useful to achieve locoregional delivery to a tumor, including a catheter itself, or may comprise a catheter along with other components (e.g. filter valve, balloon, pressure sensor system, pump system, syringe, outer delivery catheter, etc.) that may be used in combination with the catheter. In certain embodiments, the catheter is a microcatheter.

In some embodiments, the device may have one or more attributes that include, but are not limited to, self-centering capability that can provide homogeneous distribution of therapy in downstream branching network of vessels; anti-reflux capability that can block or inhibit the retrograde flow of the TLR agonist (for example, with the use of a valve and filter, and/or balloon); a system to measure the pressure inside the vessel; and a means to modulate the pressure inside the vessel, such as by causing a decrease in pressure at placement and during the TLR agonist infusion, and an increase of pressure during saline bolus or during bolus infusion of the TLR agonist. In some embodiments, the system is designed to continuously monitor real-time pressure or flow throughout the procedure.

In some embodiments, the device that may be used to perform the methods of the present invention is a device as disclosed in U.S. Pat. Nos. 8,500,775, 8,696,698, 8,696,699, 9,539,081, 9,808,332, 9,770,319, 9,968,740, 10,813,739, 10,588,636, 11,090,460, U.S. Patent Publication No. 2018/0193591, U.S.

Patent Publication No. 2018/0250469, U.S. Patent Publication No. 2019/0298983, U.S. Patent Publication No. 2020/0038586, and U.S. Patent Publication No. 2020-0383688, which are all incorporated by reference herein in their entireties.

In some embodiments, the device is a device as disclosed in U.S. Pat. No. 9,770,319. In certain embodiments, the device may be a device known as the Surefire Infusion System.

In some embodiments, the device supports the measurement of intravascular pressure during use. In some embodiments, the device is a device as disclosed in U.S. Patent Publication No. 2020-0383688. In certain embodiments, the device may be a device known as the TriSalus Infusion System. In certain embodiments, the device may be a device known as the TriNav® Infusion System. The TriNav® is a single lumen catheter equipped with a one-way valve that responds dynamically to local pressure and flow changes, such as those arising from the cardiac cycle or generated by infusion. The valve structure modulates distal vascular pressures and blood flow. This in turn may alter therapeutic distribution and first-pass absorption due to increased contact time within the vasculature.

In some embodiments, the TLR agonist may be administered through a device via PEDD. In some embodiments, the TLR agonist may be administered while monitoring the pressure in the vessel, which can be used to adjust and correct the positioning of the device at the infusion site and/or to adjust the rate of infusion. Pressure may be monitored by, for example, a pressure sensor system comprising one or more pressure sensors.

The rate of infusion may be adjusted to alter vascular pressure or flow, which may promote the penetration and/or binding of the TLR agonist into the target tissue or tumor or at its surface. In some embodiments, the rate of infusion may be adjusted and/or controlled using a syringe pump as part of the delivery system or by any other method (e.g., an infusion flow rate regulating device). In some embodiments, the rate of infusion may be adjusted and/or controlled using a pump system. In some embodiments, the rate of infusion using a pump system may be about 0.1 cc/min to about 40 cc/min, or about 0.1 cc/min to about 30 cc/min, or about 0.5 cc/min to about 25 cc/min, or about 0.5 cc/min to about 20 cc/min, or about 1 cc/min to about 15 cc/min, or about 1 cc/min to about 10 cc/min, or about 1 cc/min to about 8 cc/min, or about 1 cc/min to about 5 cc/min. Further, the rate of infusion using a bolus infusion may be about 30 cc/min to about 360 cc/min, or about 120 cc/min to about 240 cc/min. In one embodiment the SD-101 infusion procedure lasts approximately 10-200 minutes. In another embodiment the SD-101 infusion procedure lasts approximately 10-60 minutes. In another embodiment the SD-101 infusion procedure lasts approximately 25 minutes.

### Methods Comprising Administration to the Liver

In an embodiment, the methods of the present invention include methods of treating a solid tumor in the liver, such as a tumor that is the result of at least one primary liver cancer such as HCC and ICC or a tumor having features of both HCC and ICC, said method comprising administering a toll-like receptor agonist to a patient in need thereof, wherein the toll-like receptor agonist is administered through a device by HAI to such solid tumor in the liver. HAI refers to the infusion of a treatment into the hepatic artery of the liver or branches of the hepatic artery. According to an embodiment, the toll-like receptor agonist or agonists are introduced through the percutaneous introduction of a device into the branches of a hepatic artery or portal vein, such as a catheter and/or a device that facilitates pressure-enabled delivery. According to an embodiment, the toll-like receptor agonist is a TLR9 agonist and in some embodiments the TLR9 agonist is SD-101. In one embodiment, the patient is a human patient. According to another embodiment, the methods include administration to a subject who is male or female, and is eighteen years of age or older.

According to another embodiment, the methods of the present invention include methods of treating a solid tumor in the liver, such as a tumor that is the result of at least one primary liver cancer such as HCC and ICC or a tumor having features of both HCC and ICC, said method comprising administering a toll-like receptor agonist to a patient in need thereof, wherein the toll-like receptor agonist is administered through a device by PVI to such solid tumor in the liver. PVI refers to the infusion of a treatment into the hepatic portal venous system. According to an embodiment, the toll-like receptor agonist or agonists are introduced through the percutaneous introduction of a device into the branches of the hepatic portal venous system, such as a catheter and/or a device that facilitates pressure-enabled delivery. According to an embodiment, the toll-like receptor agonist is a TLR9 agonist and in some embodiments the TLR9 agonist is SD-101. In one embodiment, the patient is a human patient.

According to another embodiment, the methods of the present invention include a method for treating at least one primary liver cancer such as HCC and ICC or a tumor having features of both HCC and ICC, wherein the subject has not received prior cytotoxic chemotherapy, targeted therapy, or external radiation therapy within 14 days prior to enrollment. According to yet another embodiment, methods of the present invention are administered to subject who have not ever received therapy with SD-101. According to an embodiment of the invention, methods also include administration to subject who have not ever received prior embolic HAI therapy with permanent embolic material.

According to another embodiment, methods of the present invention include a method for treating at least one primary liver cancer such as HCC and ICC or a tumor having features of both HCC and ICC, wherein the subject has no prior history of or other concurrent malignancy unless the malignancy is clinically insignificant. In another embodiment, subjects who are treated according to the methods of the present invention may have no ongoing treatment. In yet another embodiment, the subject is clinically stable.

In another embodiment, methods of the present invention may include administration to a subject who has measurable disease in the liver according to RECIST v.1.1 criteria. In an additional embodiment, methods of the present invention may include administration to a subject who exhibits an Eastern Cooperative Oncology Group (ECOG) performance score (PS) of 0-1 at screening. In another embodiment, subjects who are administered therapy according to methods of the present invention have a life expectancy of greater than 3 months at screening as estimated by the investigator. In yet another embodiment, subjects have a QTc interval≤480 msec.

In another embodiment, all associated clinically significant drug-related toxicity from previous cancer therapy is resolved prior to treatment. In this embodiment, resolution is to Grade≤1 or the patient's pretreatment level. In an additional embodiment, the subject may have Grade 2 alopecia and endocrinopathies controlled on replacement therapy.

In another embodiment, methods of the present invention may include administration to a subject who has adequate organ function at screening. In an embodiment, a subject with adequate organ function may exhibit one or more of the following: (i) platelet count >100,000/μL, (2) hemoglobin ≥8.0 g/dL, (3) white blood cell count (WBC)>2,000/μL (4) Serum creatinine≤2.0 mg/dL unless the measured creatinine clearance is ≥30 mL/min calculated by Cockcroft-Gault formula, (5) total and direct bilirubin≤2.0× the upper limit of normal (ULN) and alkaline phosphatase≤5×ULN, (6) for patients with documented Gilbert's disease, total bilirubin up to 3.0 mg/dL, (7) ALT and AST≤5×ULN, and (8) prothrombin time/International Normalized Ratio (INR) or activated partial thromboplastin time (aPTT) test results at screening ≤1.5×ULN (this applies only to patients who do not receive therapeutic anticoagulation; patients receiving therapeutic anticoagulation should be on a stable dose for at least 4 weeks prior to the first dose of study intervention).

According to one or more embodiments, the methods of the present invention include methods for treating a primary liver cancer, such as HCC or ICC, wherein the subject has histologically or cytologically confirmed HCC or ICC with liver-only or liver-dominant disease. Liver-dominant disease may present with intrahepatic disease representing the largest fraction of disease, or if progression of HCC or ICC represents a significant threat to the patient's life.

In one or more embodiments, methods of the present invention may include administration to a subject who has a designation of class A on the Child-Pugh liver function scale (a three-category scale [A, B, or C], with C indicating the most severe compromise of liver function). In one or more additional embodiments, methods of the present invention may include administration to a subject with adequate hematologic and organ function.

In one or more embodiments, methods of the present invention may include administration to a subject who previously received at least 1 standard line of systemic therapy for liver cancer and with persistent or progressive measurable disease, as defined by RECIST version 1.1, that is not amenable to curative therapies.

According to another embodiment, the tumor is unresectable.

According to another embodiment, the methods of the present invention can be administered with other cancer therapeutics such as immuno-modulators, tumor-killing agents, and/or other targeted therapeutics.

According to an embodiment, TLR9 therapy may be administered in combination with cell therapy (thereby enabling cell therapy by modulation of the immune system), chemoembolic treatment, or radioembolic treatment.

In one embodiment, the above methods of administration to the liver are intended to result in the penetration of the toll-like receptor agonist throughout the solid tumor, throughout the entire organ, or substantially throughout the entire tumor. In an embodiment, such methods enhance perfusion of the toll-like receptor agonist to a patient in need thereof, including by overcoming interstitial fluid pressure and solid stress of the tumor. In another embodiment, perfusion throughout an entire organ or portion thereof, may provide benefits for the treatment of the disease by thoroughly exposing the tumor to therapeutic agent. In an embodiment, such methods are better able to afford delivery of the toll-like receptor to areas of the tumor that have poor access to systemic circulation. In another embodiment, such methods deliver higher concentrations of the toll-like receptor agonist into such a tumor with less toll-like receptor agonist delivered to nontarget tissues compared to conventional systemic delivery via a peripheral vein. Nontarget tissues are tissues directly perfused by the arterial network in immediate connection with the infusion device. In one embodiment, such methods result in the reduction in size, reduction in growth rate, or shrinkage or elimination of the solid tumor.

The methods of the present invention may also include mapping the vessels leading to the right and left lobes of the liver prior to performing HAI, or selective infusion into specific sectors or segments, and when necessary, occluding vessels that do not lead to the liver or that are otherwise not a target. In some embodiments, prior to infusion, patients can undergo a mapping angiogram, e.g. via a common femoral artery approach.

Methods for mapping vessels in the body and delivery of therapeutics are well known to the ordinarily skilled artisan. Occlusion may be achieved, for example, through the use of microcoil embolization, which allows the practitioner to block off-target arteries or vessels, thereby optimizing delivery of the modified cells to the liver. Microcoil embolization can be performed as needed, such as prior to administering the first dose of TLR9 agonists to facilitate optimal infusion of a pharmaceutical composition comprising the TLR9 agonists. In another embodiment, a sterile sponge (e.g. GELFOAM) can be used. In this regard, the sterile sponge can be cut and pushed into the catheter. In another embodiment, the sterile sponge can be provided as granules.

In some embodiments, doses of a TLR9 agonist, such as SD-101 may be about 0.01 mg, about 0.03 mg, about 0.05 mg, about 0.1 mg, about 0.3 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, or about 8 mg. In some embodiments, SD-101 is administered at doses of 12 mg, 16 mg, and 20 mg. Administration of a milligram amount of SD-101 (e.g. about 2 mg) describes administering about 2 mg of the composition illustrated in FIG. 1. For example, such an amount of SD-101 (e.g. about a 2 mg amount) may also exist within a composition that contains material in addition to such amount of SD-101, such as other related and unrelated compounds. Equivalent molar amounts of other pharmaceutically acceptable salts are also contemplated.

In some embodiments, doses of a TLR9 agonist, such as SD-101, may be between about 0.01 mg and about 20 mg, about 0.01 mg and about 10 mg, between about 0.01 mg and about 8 mg, and between about 0.01 mg and about 4 mg. In some embodiments, doses of a TLR9 agonist, such as SD-101 may be between about 2 mg and about 10 mg, between about 2 mg and about 8 mg, and between about 2 mg and about 4 mg. In some embodiments, doses of a TLR9 agonist, such as SD-101 may be less than about 10 mg, less than about 8 mg, less than about 4 mg, or less than about 2 mg. Such doses may be administered daily, weekly, or every other week. In one embodiment, doses of SD-101 are incrementally increased, such as through administration of about 2 mg, followed by about 4 mg, and then followed by about 8 mg.

In some embodiments, the methods of the present invention may comprise administering a dosing regimen comprising cycles, in which one or more of the cycles comprise administering SD-101 via HAI and PEDD. As used herein, a “cycle” is a repeat of a dosing sequence. In one embodiment, one cycle comprises three weekly doses per cycle (i.e. administration of SD-101 once per week over three consecutive weeks). In one embodiment, a cycle of treatment according to the present invention may comprise periods of SD-101 administration followed by “off” periods or rest periods. In another embodiment, in addition to three weekly doses per cycle, the cycle further comprises one week, two weeks, three weeks, or four weeks as a rest period following the weekly administration of SD-101. In yet another embodiment, in addition to three weekly doses per cycle, the cycle further comprises about thirty-eight days as a rest period following the weekly administration of SD-101. In another embodiment, the entire cycle comprises about fifty-two days. In another embodiment, the dosing regimen comprises at least one, at least two, or at least three cycles, or longer.

In some embodiments, the present invention relates to the use of a TLR9 agonist in the manufacture of a medicament for treating a solid tumor in the liver, such as a tumor that is the result of a primary liver cancer such as HCC and ICC, said method comprising administering the TLR9 agonist to a patient in need thereof, wherein the TLR9 agonist is administered through a device by HAI to such solid tumor in the liver.

In some embodiments, SD-101 is administered for the treatment of primary liver cancers such as HCC and ICC at a dose of 0.5 mg through HAI, and in some embodiments, the SD-101 is further administered through a device that modulates pressure (i.e. PEDD). In some embodiments, SD-101 is administered at a dose of 0.5 mg through HAI through a device that modulates vascular pressure in combination with a CPI, wherein the CPI is nivolumab. In other embodiments, SD-101 is administered at a dose of 0.5 mg through HAI and through a device that modulates pressure in combination with ipilimumab. In some embodiments, SD-101 is administered at a dose of 0.5 mg through HAI and through a device that modulates pressure in combination with ipilimumab and nivolumab. In other embodiments, SD-101 is administered at a dose of 0.5 mg through HAI and through a device that modulates pressure in combination with pembrolizumab.

In some embodiments, SD-101 is administered for the treatment of primary liver cancers such as HCC and ICC at a dose of 2 mg through HAI, and in some embodiments, the SD-101 is further administered through a device that modulates pressure (i.e. PEDD). In some embodiments, SD-101 is administered at a dose of 2 mg through HAI through a device that modulates vascular pressure in combination with a CPI, wherein the CPI is nivolumab. In other embodiments, SD-101 is administered at a dose of 2 mg through HAI and through a device that modulates pressure in combination with ipilimumab. In some embodiments, SD-101 is administered at a dose of 2 mg through HAI and through a device that modulates pressure in combination with ipilimumab and nivolumab. In other embodiments, SD-101 is administered at a dose of 2 mg through HAI and through a device that modulates pressure in combination with pembrolizumab.

In some embodiments, SD-101 is administered for the treatment of a primary liver cancer such as HCC and ICC at a dose of 4 mg through HAI, and in some embodiments, the SD-101 is further administered through a device that modulates pressure (i.e. PEDD). In some embodiments, SD-101 is administered at a dose of 4 mg through HAI through a device that modulates vascular pressure in combination with a CPI, wherein the CPI is nivolumab. In other embodiments, SD-101 is administered at a dose of 4 mg through HAI and through a device that modulates pressure in combination with ipilimumab. In some embodiments, SD-101 is administered at a dose of 4 mg through HAI and through a device that modulates pressure in combination with ipilimumab and nivolumab. In other embodiments, SD-101 is administered at a dose of 4 mg through HAI and through a device that modulates pressure in combination with pembrolizumab.

In some embodiments, SD-101 is administered for the treatment of a primary liver cancer such as HCC and ICC at a dose of 8 mg through HAI, and in some embodiments, the SD-101 is further administered through a device that modulates pressure (i.e. PEDD). In some embodiments, SD-101 is administered at a dose of 8 mg through HAI through a device that modulates vascular pressure in combination with a CPI, wherein the CPI is nivolumab. In other embodiments, SD-101 is administered at a dose of 8 mg through HAI and through a device that modulates pressure in combination with ipilimumab. In some embodiments, SD-101 is administered at a dose of 8 mg through HAI and through a device that modulates pressure in combination with ipilimumab and nivolumab. In other embodiments, SD-101 is administered at a dose of 8 mg through HAI and through a device that modulates pressure in combination with pembrolizumab.

In one or more embodiments, methods of the present invention may include administering up to 6 doses (maximum of 2 cycles of SD-101, 3 weekly doses per cycle) to a subject. In one or more embodiments, fewer doses or cycles of SD-101 may be administered based on toxicity or tolerability of the subject.

In one or more embodiments, a solution of SD-101 may be administered to a subject via HAI using a PEDD device, such as the TriNav®. In some such embodiments, vascular access may be achieved using the femoral artery or radial artery approach. Hemangiomata, shunting vessels, or other vascular lesions in the liver that may interfere with therapeutic delivery may be embolized at the discretion of the treating interventional radiology specialist. In one or more embodiments, the SD-101 can be prepared and delivered in a 50 ml syringe (therapeutic dose) and a 100-mL vial containing the volume necessary for the therapeutic flush (10 mL), both at the therapeutic concentration. The PEDD device can then be advanced into the target vessels.

In one or more embodiments, the 50 mL solution of SD-101 can be allocated by per segment or sector of the liver. In one or more embodiments, the 50-mL therapeutic dose of SD-101 can be allocated as follows: 3×10 mL infusions into target blood vessels in the right hepatic lobe and 2×10 mL infusions into target blood vessels in the left hepatic lobe. Further, the distribution of the 10-mL aliquots may be adjusted based on the location of measurable disease and target vessel diameter. In one or more embodiments, the SD-101 infusion can be expected to last approximately 10-60 minutes. For example, in some embodiments, the infusion time can be approximately 25 minutes. Further, in another embodiment, the overall interventional procedure can last between 30-80 minutes. This involves all the handling time between infusions in different locations. In some embodiments, the 50 mL solution of SD-101 can include one of 0.5 mg, 2 mg, 4 mg, or 8 mg of SD-101. In this regard, the infused dose of SD-101 can be one of 0.01 mg/mL, 0.04 mg/mL, 0.08 mg/mL, or 0.16 mg/mL.

According to another embodiment, the SD-101 can be prepared and delivered in a 25 mL solution. In some embodiments, the 25 mL solution of SD-101 can include one of 0.5 mg, 2 mg, 4 mg, or 8 mg of SD-101. In this regard, the infused dose of SD-101 can be one of 0.02 mg/mL, 0.08 mg/mL, 0.16 mg/mL, or 0.32 mg/mL.

According to another embodiment, the SD-101 can be prepared and delivered in a 10 mL solution. In some embodiments, the 10 mL solution of SD-101 can include one of 0.5 mg, 2 mg, 4 mg, or 8 mg of SD-101. In this regard, the infused dose of SD-101 can be one of 0.05 mg/mL, 2 mg/mL, 4 mg/mL, or 0.8 mg/mL.

In some embodiments, the methods of the present invention result in the treatment of target lesions. In this embodiment, the methods of the present invention may result in a complete response, comprising the disappearance of all target lesions. In some embodiments, the methods of the present invention may result in a partial response, comprising at least a 30% decrease in the sum of the longest diameter of target lesions, taking as reference the baseline sum longest diameter. In some embodiments, the methods of the present invention may result in stable disease of target lesions, comprising neither sufficient shrinkage to qualify for partial response nor sufficient increase to qualify for progressive disease, taking as reference the smallest sum longest diameter since the treatment started. In such an embodiment, progressive disease is characterized by at least a 20% increase in the sum of the longest diameter of target lesions, taking as reference the smallest sum longest diameter recorded since the treatment started or the appearance of one or more new lesions. The sum must demonstrate an absolute increase of 5 mm.

In another embodiment, the methods of the present invention result in the treatment of nontarget lesions. Nontarget lesions are lesions not directly perfused by the arterial network in immediate communication with the infusion system. In this embodiment, the methods of the present invention may result in a complete response, comprising the disappearance of all nontarget lesions. In some embodiments, the methods of the present invention result in persistence of one or more nontarget lesion(s), while not resulting in a complete response or progressive disease. In such an embodiment, progressive disease is characterized by unequivocal progression of existing nontarget lesions, and/or the appearance of one or more new lesions.

In some embodiments, the methods of the present invention result in a beneficial overall response rate, such as an overall response rate according to RECIST v.1.1. In those embodiments, the methods of the present invention result in an overall response that is a complete response wherein the subject exhibits a complete response of target lesions, a complete response of nontarget lesions, and no new lesions. In other embodiments, the methods of the present invention result in an overall response that is a partial response, wherein the subject exhibits a complete response for target lesions, non-complete response and non-progressive disease for nontarget lesions, and no new lesions. In other embodiments, the methods of the present invention result in an overall response that is a partial response, wherein the subject exhibits a partial response for target lesions, non-progressive disease for nontarget lesions, and no new lesions. In another embodiment, the methods of the present invention result in an overall response that is stable disease wherein the subject exhibits stable disease of target lesions, non-progressive disease for nontarget lesions, and no new lesions.

In some embodiments, the methods of the present invention result in an increased duration of overall response. In some embodiments, the duration of overall response is measured from the time measurement criteria are met for complete response or partial response (whichever is first recorded) until the first date that recurrent or progressive disease is objectively documented (taking as reference for progressive disease the smallest measurements recorded since the treatment started). The duration of overall complete response may be measured from the time measurement criteria are first met for complete response until the first date that progressive disease is objectively documented. In some embodiments, the duration of stable disease is measured from the start of the treatment until the criteria for progression are met, taking as reference the smallest measurements recorded since the treatment started, including the baseline measurements.

In yet other embodiments, the methods of the present invention result in improved overall survival rates. For example, overall survival may be calculated from the date of enrollment to the time of death. Patients who are still alive prior to the data cutoff for final efficacy analysis, or who dropout prior to study end, will be censored at the day they were last known to be alive.

In other embodiments, the methods of the prevent invention result in progression-free survival. For instance, progression-free survival may be calculated from the date of enrollment to the time of CT scan documenting relapse (or other unambiguous indicator of disease development), or date of death, whichever occurs first. Patients who have no documented relapse and are still alive prior to the data cutoff for final efficacy analysis, or who drop out prior to study end, will be censored at the date of the last radiological evidence documenting absence of relapse.

In some embodiments, the methods of the present invention result in a beneficial overall response rate, such as an overall response rate according to mRECIST. In those embodiments, the methods of the present invention result in an overall response that is a complete response wherein the subject exhibits a complete response of target lesions, a complete response of nontarget lesions, and no new lesions. In other embodiments, the methods of the present invention result in an overall response that is a partial response, wherein the subject exhibits a complete response for target lesions, non-complete response and incomplete response for nontarget lesions, and no new lesions. In other embodiments, the methods of the present invention result in an overall response that is a partial response, wherein the subject exhibits a partial response for target lesions, non-progressive disease for nontarget lesions, and no new lesions. In another embodiment, the methods of the present invention result in an overall response that is stable disease wherein the subject exhibits stable disease of target lesions, non-progressive disease for nontarget lesions, and no new lesions.

In some embodiments, the methods of the present invention result in a beneficial overall response rate, such as an overall response rate according to iRECIST.

According to another embodiment, the methods of the present invention include a method for treating a primary liver cancer such as HCC and ICC, wherein the administration of SD-101 results in a reduction of tumor burden. In some embodiments, the tumor burden is reduced by about 10%, by about 20%, by about 30%, by about 40%, by about 50%, by about 60%, by about 70%, by about 80%, by about 90%, or by about 100%.

According to another embodiment, the methods of the present invention include a method for treating a primary liver cancer such as HCC and ICC, wherein the administration of SD-101 results in a reduction of tumor progression or stabilization of tumor growth. In some embodiments, tumor progression is reduced by about 10%, by about 20%, by about 30%, by about 40%, by about 50%, by about 60%, by about 70%, by about 80%, by about 90%, or by about 100%.

According to another embodiment, the methods of the present invention include a method for treating a primary liver cancer such as HCC and ICC, wherein the administration of SD-101 reprograms the liver MDSC compartment to enable immune control of the liver cancer and/or improves responsiveness to systemic anti-PD-1 therapy through elimination of MDSC. In some embodiments, the methods of the present invention are superior in controlling MDSC. In some embodiments, the methods of the present invention include a method for treating primary liver cancers such as HCC and ICC, wherein the administration of SD-101 reduces the frequency of MDSC cells (CD11b+Gr1+), monocytic MDSC (M-MDSC; CD11b+Ly6C+) cells, granulocytic MDSC (G-MDSC; CD11b+LY6G+) cells, or human MDSC (CD33+CD11b+HLADR− (CD14+ for m-MDSC and CD15+ for G-MDSC)). According to another embodiment, the methods of the present invention enhance M1 macrophages (CD14+CD86+). According to yet another embodiment, the methods of the present invention decrease M2 macrophages (CD14+CD163+CD206+)

In another embodiment, the methods of the present invention increase NFκB activation. In yet an additional embodiment, the methods of present invention increase IL-6. In another embodiment, the methods of the present invention increase IL-10. In yet an additional embodiment, the methods of present invention increase IL-29. In another embodiment, the methods of the present invention increase IFNα. As a further embodiment, the methods of the present invention decrease STAT3 phosphorylation.

The present invention will be further illustrated and/or demonstrated in the following Examples, which is given for illustration/demonstration purposes only and is not intended to limit the invention in anyway.

### Example 1

The objective of this study was to evaluate the distribution of an infused TLR9 agonist, SD-101, in and around a primary liver tumor. In particular, the study was conducted on a novel swine model that forms liver tumors in situ. To recapitulate primary liver tumor development, biopsies of liver tissue were collected from the swine and then the cells were exposed to a vector to induce cancerous growth. These cells were subsequently engrafted into the liver tissue by local needle injection and allowed to develop tumors approximately 2 cm in diameter.

The TLR9 agonist SD-101 sequence oligo was synthesized and conjugated to the IRD800CW (ex. 767 nm, em. 791 nm) fluorophore. Then, the IRD800CW-SD-101 (1 ml at 2.5 nmol) was mixed with SD-101 (0.3 ml, containing 4 mg SD-101 drug product) into 9 ml of sterile saline solution to produce a 10.3 ml total volume for infusion. The solution was infused via HAI using a PEDD device, i.e., the TriNav®.

The Seldinger technique was used to gain access through the femoral artery of the subject swine. A 5F introducer sheath was secured at the site. A 5F angiographic catheter was used to perform angiography to identify hepatic arterial anatomy. A 1.5- to 3.5-mm diameter vessel feeding the entire left lateral, left medial, right medial, or right lateral lobe containing the tumor was then selected. The TriNav® was then tracked into the target vessel location.

The syringe containing the solution was then placed into a syringe pump and delivered at a rate of 2 ml/min through the TriNav®. After the first 4 ml of solution were infused, a 1 ml high-pressure bolus was conducted at a rate of 2 cc/sec for a duration of 0.5 seconds. The remainder of the solution was then administered at 2 ml/min.

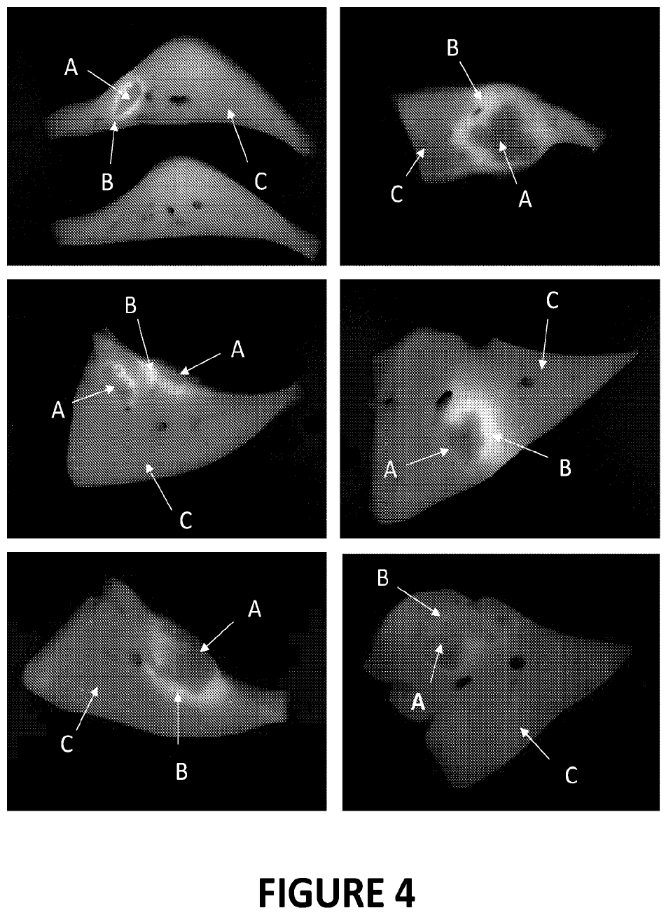

Normal physiologic conditions were maintained for 60 minutes, after which time the swine were euthanized and their livers were removed. Each lobe of the liver was separated and near-IR imaging with a Pearl Trilogy Imaging System was performed to identify patterns of drug uptake. Each lobe was then cut into 1 cm thick sections and separately imaged (85 μm resolution, 700 nm, 800 nm, and white light channels) on both tissue faces using the Pearl system. The full liver volume was analyzed to visualize the distribution of fluorescently-labeled SD-101, as depicted in FIG. 4. In this regard, A depicts the regions of tumor tissue and necrosis, B depicts the regions of growing primary tumor and surrounding normal tissue with high uptake of fluorescently-labeled SD-101, and C depicts normal tissue (which shows little to no labeled SD-101 fluorescence signal).

The study demonstrated that pressure-enabled delivery of labeled SD-101 using a TriNav® resulted in the preferential accumulation of fluorescent compound in the region around the actively growing primary liver tumor periphery and surrounding normal tissue, in which the region is expected to contain the bulk of immune cells expressing TLR9, which is the target of SD-101. Further, infusions were conducted through vessels feeding an entire lobe containing a primary tumor, with most of the tissue in the perfused territory being normal liver. Despite not infusing SD-101 through specific tumor feeding vessels, the use of PEDD resulted in preferential distribution of SD-101 in and around the tumor, with relatively low drug exposure in normal liver tissue.

### Example 2

In this example, infusions of a TLR9 agonist, SD-101, were administered with the aim of enhancing response rates to CPI therapy in patients with primary liver cancers such as HCC and ICC. SD-101 is a TLR9 agonist that can stimulate a variety of immune cells, in addition to favorably eliminating or reprogramming suppressive immune cells that drive tumor progression in the liver. Pressure-Enabled Drug Delivery (PEDD) via hepatic artery infusion (PEDD/HAI) will be applied in this study to enable effective delivery of SD-101 to HCC tumors, while limiting systemic exposure.

The study has two phases, i.e. Phases 1b and 2. In this regard, the primary objective for Phase 1b is to determine the safety of and to identify the maximum tolerated dose (MTD) or optimal dose of PEDD/HAI of SD-101 alone, the MTD or optimal dose of SD-101 in combination with pembrolizumab or both nivolumab and ipilimumab in patients with HCC or ICC or a mixture of both HCC and ICC. Further, the secondary objective is to assess preliminary efficacy in terms of RECIST for immune based therapeutics (iRECIST) ORR, modified RECIST (mRECIST) ORR, RECIST 1.1 hepatic-specific response rate (HRR), overall progression-free survival (PFS), and clinical benefit (complete response [CR]+partial response [PR]+stable disease [SD]). With regard to Phase 2, the primary objective is to assess the Response Evaluation Criteria in Solid Tumors (RECIST) v1.1 overall response rate (ORR) to PEDD/HAI of SD-101 in combination with intravenous (IV) immune checkpoint blockade. The checkpoint regimen used in combination with SD-101 will be chosen based on safety and response data from Phase 1b. Separate cohorts for HCC and ICC will be enrolled. Further, the secondary objective is to assess the duration of response (DOR), 12-month overall survival (OS), and safety/toxicity of the chosen MTD or optimal dose of SD-101 in combination with CPI. Further, there are also exploratory objectives: (i) to assess RECIST v1.1 hepatic-specific progression-free survival (HPFS); (ii) to assess pathologic response following PEDD/HAI of SD-101 with or without systemic IV CPI infusion and correlation with imaging response scoring; (iii) to assess the intratumoral immunological effects of treatment on MDSC, lymphocytes, and cytokine profiles using paired baseline and on-treatment liver tumor and normal liver biopsies; (iv) to assess the peripheral immunological pharmacodynamic effects of treatment using serial blood sampling for circulating tumor cells (CTC), circulating cytokines, and other immunologic correlatives; (v) to assess changes from baseline in ECOG PS over time; (vi) to assess changes from baseline in quality of life using the European Organization for the Research and Treatment of Cancer Quality of Life Questionnaire for Cancer (EORTC-QLQ-C30) instrument.

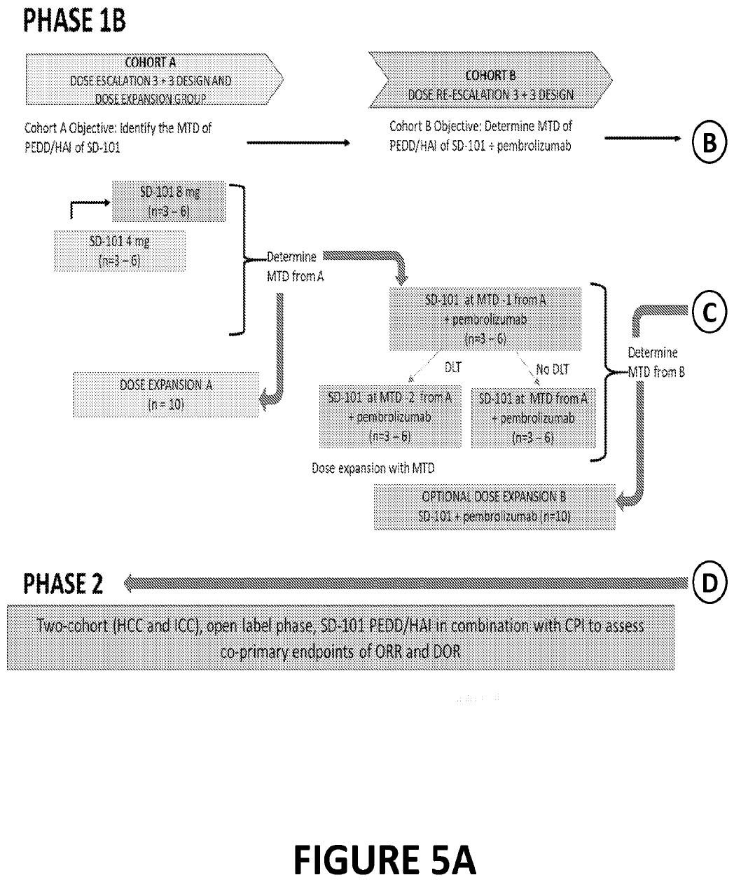

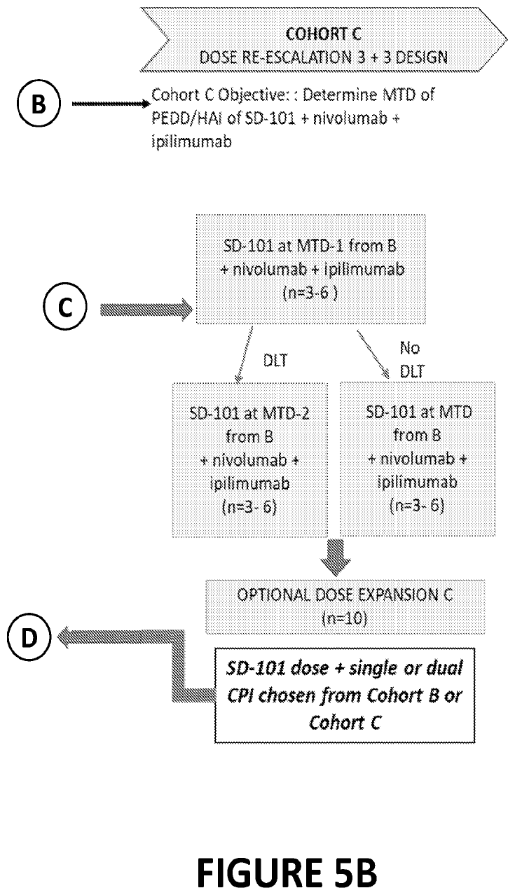

The overall design for the study can be found in FIG. 5. The study is open-label, multicenter, and nonrandomized.

As described in further detail below, in Phase 1b, escalating doses of SD-101 can be administered alone (Cohort A), together with pembrolizumab (Cohort B), or together with combined ipilimumab and nivolumab (Cohort C). Cohorts B and C will begin one dose level below the MTD or optimal dose from Cohort A to optimize safety when adding CPI to SD-101. Cohort B will not begin until completion of the DLT window for the final patient in Cohort A (Cohort B will proceed concurrently with the Cohort A expansion). Cohort C will not begin until completion of the DLT window for the final patient in Cohort B. A standard 3+3 dose-escalation design will be employed to determine the MTD.

Following the first SD-101 infusion for each patient in Phase 1b, an overnight in-hospital observation or admission is required. If the first SD-101 dose is well tolerated, further overnight observation or admission is at the discretion of the treating physician for subsequent SD-101 infusions. If subsequent infusions are performed on an outpatient basis, the patient will be observed for a minimum of 6 hours post infusion before being discharged, if clinically stable. If there are any events >Grade 2 related to SD-101 PEDD/HAI that required in-patient therapy following the first infusion, the patient will be kept for overnight observation or admission following each subsequent SD-101 infusion.

Following determination of the recommended MTD or optimal dose of SD-101 for PEDD/HAI and which CPI regimen(s) are tolerated, the study will progress to Phase 2 to assess efficacy. Patients in Phase 2 will receive the SD-101 dose selected from Phase 1b together with systemic single- or double-agent checkpoint blockade. The choice of single or dual CPI therapy together with SD-101 for Phase 2 will consider safety data in addition to response rates from Cohorts B and C in Phase 1b. SD-101 will be administered over 2 cycles, with 3 weekly doses per cycle.

### Inclusion Criteria

According to an embodiment, to be included in the study, the patient must meet all of the following criteria for inclusion:
1. 18 years of age or older with locally advanced, metastatic or unresectable hepatocellular carcinoma or intrahepatic cholangiocarcinoma, with the diagnosis confirmed by histologic or cytologic analysis or clinical features according to the American Association for the Study of Liver Diseases.
2. Previously received at least 1 standard line of systemic therapy for liver cancer and with persistent or progressive measurable disease, as defined by RECIST version 1.1, that is not amenable to curative therapies.
3. Has performance status score of 0 or 1 on the ECOG scale (scores range from 0 to 5, with higher numbers reflecting greater disability).
4. Designation of class A on the Child-Pugh liver function scale (a three-category scale [A, B, or C], with C indicating the most severe compromise of liver function).
5. Adequate hematologic and organ function.
6. Has histologically or cytologically confirmed HCC or ICC with liver-only or liver-dominant disease. Liver-dominant will be defined as intrahepatic disease representing the largest fraction of disease.
7. Able to understand the study and provide written informed consent prior to any study procedures.
8. Has not received prior cytotoxic chemotherapy, targeted therapy, or external radiation therapy within 14 days prior to screening.
9. Has not ever received prior embolic HAI therapy with permanent embolic material. Note: Previous embolic HAI therapy with permanent embolic material will not be exclusionary if following this therapy, the target vessels are not occluded, and tumors are perfused based on the patient's screening.
10. Has no prior history of or other concurrent malignancy unless the malignancy is clinically insignificant, no ongoing treatment is required, and the patient is clinically stable.
11. Has measurable disease in the liver according to RECIST v.1.1 criteria.
12. Has a life expectancy of >3 months at screening as estimated by the investigator.
13. Has a QTc interval≤480 msec.
14. All associated clinically significant (in the judgment of the investigator) drug-related toxicity from previous cancer therapy must be resolved (to Grade≤1 or the patient's pretreatment level) prior to study treatment administration (Grade 2 alopecia and endocrinopathies controlled on replacement therapy are allowed).
15. Has adequate organ function at screening as evidence by:
   Platelet count >100,000/μL.
   Hemoglobin ≥8.0 g/dL.
   White blood cell count (WBC)>2,000/μL.
   Serum creatinine≤2.0 mg/dL unless the measured creatinine clearance is ≥30 mL/min calculated by Cockcroft-Gault formula.
   Total and direct bilirubin≤2.0× the upper limit of normal (ULN) and alkaline phosphatase≤5×ULN. For patients with documented Gilbert's disease, total bilirubin up to 3.0 mg/dL is allowed.
   ALT and AST≤5×ULN.
   Prothrombin time/International Normalized Ratio (INR) or activated partial thromboplastin time (aPTT) test results at screening≤1.5×ULN (this applies only to patients who do not receive therapeutic anticoagulation; patients receiving therapeutic anticoagulation should be on a stable dose for at least 4 weeks prior to the first dose of study intervention).
   Note: Laboratory tests with exclusionary results judged by the investigator as not compatible with the patient's clinical status may be repeated once for eligibility purposes.
16. Females of childbearing potential must be nonpregnant and nonlactating, or post-menopausal, and have a negative serum human chorionic gonadotropin (hCG) pregnancy test result at screening and a negative urine or serum pregnancy test prior to the first dose of study intervention.
   Females of childbearing potential must agree to abstain from sexual activity with nonsterilized male partners, or if sexually active with a nonsterilized male partner must agree to use highly effective methods of contraception from screening, throughout the study and agree to continue using such precautions for 100 days after the final dose of study intervention.
   Nonsterilized males who are sexually active with a female of childbearing potential must agree to use effective methods of contraception and avoid sperm donation from Day 1 throughout the study and for 30 days after the final dose of study intervention.

### Phase 1b

The separate Phase 1b patient cohorts are as follows:

Cohort A—Dose-escalation cohort of PEDD/HAI of SD-101 monotherapy (2 cycles, 1 dose per week×3 weeks, per cycle) using a standard 3+3 design followed by an optional expansion group to identify the MTD or optimal dose of SD-101 alone and estimate the monotherapy response rate in HCC and ICC patients:
Dose level 1: 4 mg (n=3-6).
Dose level 2: 8 mg (n=3-6).

Enrollment of the first 2 patients at each dose level will be staggered by at least 48 hours. Additional incremental dose levels (in increments of 4 mg) may be added based on safety and response data. An optional expansion group of 10 patients at the SD-101 monotherapy MTD or optimal dose may proceed concurrently with Cohort B.

Cohort B—Standard 3+3 design dose re-escalation cohort of PEDD/HAI of SD-101 (2 cycles, 1 dose per week×3 weeks, per cycle) together with IV pembrolizumab 200 mg every 3 weeks (Q3W) to identify the MTD or optimal dose of SD-101 with single-agent pembrolizumab:
Dose level 1: Pembrolizumab together with PEDD/HAI of SD-101 at 1 dose level below the MTD or optimal dose from Cohort A (i.e., MTD-1 or optimal dose-1) (n=3-6).
Dose level 2: Pembrolizumab together with PEDD/HAI of SD-101 at the MTD or optimal dose from Cohort A (n=3-6), or if MTD-1 or optimal dose-1 not tolerated, will de-escalate to MTD-2 or optimal dose-2.

Enrollment of the first 2 patients at each dose level will be staggered by at least 48 hours. An optional expansion group of up to 10 patients receiving SD-101 together with pembrolizumab may proceed concurrently with Cohort C.

Cohort C—Standard 3+3 design dose re-escalation study of PEDD/HAI of SD-101 (2 cycles, 1 dose per week×3 weeks, per cycle) together with nivolumab 1 mg/kg in combination with ipilimumab 3 mg/kg every 3 weeks for four doses, followed by single-agent nivolumab 240 mg every 2 weeks to identify the MTD or optimal dose of SD-101 with dual-agent CPI:
Dose level 1: Nivolumab and ipilimumab together with PEDD/HAI of SD-101 at 1 dose level below the MTD or optimal dose from Cohort B (n=3-6).
Dose level 2: Nivolumab and ipilimumab together with PEDD/HAI of SD-101 at the MTD or optimal dose of SD-101 from Cohort B (n=3-6), or if MTD-1 or optimal dose-1 not tolerated, will de-escalate to MTD-2 or optimal dose-2.

Enrollment of the first 2 patients at each dose level will be staggered by at least 48 hours. An optional expansion group of 10 patients may be enrolled if additional data are needed to decide between single- and dual-agent CPI with SD-101 via PEDD/HAI for Phase 2.

### Phase 2

Two separate cohorts will be enrolled for HCC and ICC. A two-stage design will be used in Phase 2 to establish whether the proportion of responses at the SD-101 MTD or optimal dose+single- or dual-agent CPI is sufficiently high to warrant further testing. A two-stage design with the smallest total sample will be used for each tumor type.

### Duration of Administration

### Duration of SD-101 Administration (for Participants in Phase 1b and Phase 2);

Phase 1b Cohorts A. B. C. and Phase 2—Up to 6 doses (maximum of 2 cycles of SD-101, 3 weekly doses per cycle). Fewer doses or cycles of SD-101 may be administered based on toxicity or tolerability. All patients who have presence of measurable disease at baseline, one post-baseline tumor assessment, received at least 1 dose of treatment, no violation of key inclusion or exclusion criteria will be considered evaluable. However, patients who enrolled in violation of key inclusion or exclusion criteria will be considered major protocol violations and will not be considered evaluable.

### Duration of CPI Administration

Phase 1b, Cohort A:
   Not applicable.
Phase 1b, Cohort B and optional expansion cohort:
   Up to 6 months of pembrolizumab 200 mg Q3W.
Phase 1b, Cohort C and optional expansion cohort:
   IV nivolumab 1 mg/kg Q3W for 4 doses then 240 mg every 2 weeks (Q2W) for up to 12 months.
   IV ipilimumab 3 mg/kg Q3W for 4 doses.
Phase 2:
   CPI regimen as determined by Phase 1b data for up to 6 months.
Phase 1, Cohort B and optional expansion cohort:
   Up to twelve months of nivolumab 480 mg Q4W.
Phase 1, optional Cohort B1:
   Systemic IV ipilimumab 3 mg/kg Q3W for 4 doses.
Phase 1, Cohort C and optional expansion cohort:
   Systemic IV nivolumab 1 mg/kg Q3W for 4 doses then 480 mg/kg Q4W for up to twelve months and (ii) systemic IV ipilimumab 3 mg/kg Q3W for 4 doses.
Phase 1b:
   ·CPI regimen as determined by Phase 1 data for up to twelve months.

### Interventions Administered

The interventions and planned dose levels are summarized in Table 2 below.

TABLE 2

| Intervention | | Keytruda ® | Opdivo ® | | Yervoy ® |
|---|---|---|---|---|---|
| Name | SD-101 | (pembrolizumab) | (nivolumab) | | (ipilimumab) |
| Type | Drug (ODN) | Biologic (antibody) | Biologic | | Biologic |
| | | | (antibody) | | (antibody) |
| Dose | Solution | Solution for injection | Solution for | | Solution for |
| Formulation | | | injection | | injection |
| Unit Dose | 13.4 mg/mL | 100 mg/4 mL | 40 mg/4 mL | | 5 mg/mL |
| Strength(s) | | | 100 mg/10 mL | | |
| | | | 240 mg/24 mL | | |
| Dosage | 4 or 8 mg | 200 mg | 1 | mg/kg IV | 3 mg/kg IV |
| Level(s) | | | 240 | mg IV | |

| | | | | |
|---|---|---|---|---|
| Route of | Intravascular | IV | IV | IV |
| Administration^{a} | injection by | | | |
| | PEDD/HAI | | | |
| | over 10-60 | | | |
| | minutes | | | |
| Use | Experimental | Background | Background | Background |
| | | intervention | intervention | intervention |
| IMP and CPI | IMP | HCC FDA approved | HCC FDA | HCC FDA |
| | | ICC IMP | approved | approved |
| | | | ICC IMP | ICC IMP |
| Sourcing | TriSalus | Commercial - site | Commercial - site | Commercial - |
| | | supply | supply | site supply |
| Packaging and | Single-use vial^{b} | Single-dose vial | Single-dose vials | Single-dose |
| Labeling | | | | vials |

Abbreviations: HAI = hepatic artery infusion; IMP = investigational medicinal product; IV = intravenous; ODN = oligodeoxynucleotide; PEDD = Pressure-Enabled Drug Delivery device.
^{a}Each segmental, sectoral, or lobar infusion must be completed within 60 minutes to ensure that the SD-101 does not remain in the TriNav device for longer than that time. This does not limit overall procedure time.
^{c} Vial will be discarded following dose preparation for a single patient. In no instance will more than one dose be drawn from one vial.

The SD-101 solution can be infused via the hepatic arterial system using a PEDD device, e.g., the TriNav®. Vascular access may be achieved using the femoral or radial approach. Hemangiomata, shunting vessels, or other vascular lesions in the liver that may interfere with therapeutic delivery may be embolized at the discretion of the treating interventional radiology specialist. For the SD-101 infusion procedures, the drug will be prepared in the local pharmacy and delivered to the interventional radiology suite in a 50-ml syringe (therapeutic dose) and a 100-mL vial containing the volume necessary for the therapeutic flush (10-mL), both at the therapeutic concentration. The pharmacy can be notified 1 day in advance of the planned infusion time and again when the patient is on the table. The PEDD device, the TriNav®, will be advanced into the target vessels.

In one embodiment, the 50 mL volume to be administered is allocated by per segment or sector of the liver. In an embodiment, the 50-mL therapeutic dose can be allocated as follows: 3×10 mL infusions into target vessels in the right hepatic lobe and 2×10 mL infusions into target vessels in the left hepatic lobe. Further, the distribution of the 10-mL aliquots may be adjusted based on the location of measurable disease and target vessel diameter. In an embodiment, the SD-101 infusion is expected to last approximately 10-60 minutes. For example, the infusion time can be approximately 25 minutes. Further, in an embodiment, the overall interventional procedure can last between 30-80 minutes. This involves all the handling time between infusions in different locations.

### Tumor Response Evaluations

All patients will undergo imaging with magnetic resonance imaging (MRI) or CT to assess disease in the liver and other sites, as well as liver biopsies and assays of CTC, circulating cytokines, and other immunologic correlatives. Tumor response will be measured radiographically using standard RECIST v1.1 criteria. Official response scoring (per RECIST v1.1) will be preliminarily assessed on Day 84. Additional response assessments will be obtained at Day 168 to confirm earlier scoring in the event of pseudo-progression. Imaging procedures will occur every 90 days thereafter. Hepatic imaging using MRI with Eovist® contrast should be used whenever possible. Local imaging reads will be utilized for response assessment during Phase 1b and Phase 2. Independent central review for response assessment may be performed during Phase 2.

Up to 4 liver biopsies will be performed:
A baseline biopsy will be obtained on Day 1 before the first infusion of SD-101 in addition to a post infusion biopsy on Day 1. A pre infusion biopsy will be performed at the beginning of the second cycle of SD-101 (before SD-101 Infusion #4), and the final biopsy procedure occurs at Day 100.
Pathologic response will be assessed based on review by the local site pathologist with scoring of necrosis and fibrosis within tumor and normal tissue samples.

### Quality of Life

The ECOG PS scale and EORTC-QLQ-C30 questionnaire can be used to assess overall patient status and quality of life.

### Pharmacokinetics

Blood samples will be collected to characterize SD-101 systemic exposure after PEDD/HAI. No sampling or testing will be done for pembrolizumab, nivolumab or ipilimumab concentrations.
Phase 1b: Serial venous blood samples will be collected for measurement of plasma SD-101 concentrations before dosing and after the end of each dose for each dose level in Cohorts A, B, and C. Samples times may be adjusted based on results observed after initial doses for Cohort A in Phase 1b of the study.
Phase 2: If the concentrations are mostly below the lower limit of quantification of the assay in Phase 1b, these measurements may be omitted in Phase 2.

Tumor levels of SD-101 will be measured in the pre- and post-infusion biopsy specimens obtained on: Day 1 and Day 57 (pre-infusion only) for Cohorts A, B, and C.

Each plasma sample can be divided into 2 aliquots of approximately 2-3 mL each (1 for SD-101 measurement and 1 for back-up). Samples collected for analyses of SD-101 may also be used to evaluate safety or efficacy aspects related to concerns arising during or after the study.

### Pharmacodynamics

Blood samples can be collected for the measurement of CTC, circulating cytokines, and other immunologic correlatives including IFN-α and IFN-γ related gene signatures, which may be more informative than pharmacokinetic assessments for this class of therapeutic.

### Safety

Safety assessments include adverse events (AEs), clinical laboratory testing, vital signs, physical examinations, and electrocardiograms (ECGs).

The following are considered DLTs when observed during Phase 1b within 2 weeks after the last SD-101 dose in Cycle 1 and are considered attributable to study intervention (SD-101 or CPI therapy) and/or the TriNav device. Note, however, that the same adjustment or stopping rules will apply for any DLT (defined below) that may arise during the infusion period of Cycle 2:
≥Grade 3 cytokine release syndrome (CRS) per National Cancer Institute (NCI) Common Terminology Criteria for Adverse Events (CTCAE) Version 5.0.
Autoimmune AE≥Grade 3 per NCI CTCAE.
Allergic reaction AE≥Grade 3 per NCI CTCAE.
Grade 4 hematologic AE per NCI CTCAE that does not recover to ≤Grade 2 within 7 days.
Grade 4 AE per NCI CTCAE in any organ system.

Patients who develop a DLT will be permanently discontinued from study interventions. The patient can be treated according to clinical practice and monitored for resolution of the toxicity.

SD-101 and/or CPI therapy will be permanently discontinued for severe or life-threatening infusion-related reactions. Dose interruptions, delays, or discontinuation of SD-101 and/or CPI therapy is required when a patient has a Grade 3 or higher immune-mediated reaction. Discontinuation of SD-101 and/or CPI therapy for abnormal liver tests is required when a patient meets one of the conditions outlined below or in the presence of abnormal liver chemistries not meeting protocol-specified stopping rules if the investigator believes that it is in best interest of the patient.
Patient is clinically jaundiced.
Patient has evidence of coagulopathy.
Patient has clinical evidence of portal hypertension including but not limited to ascites or variceal bleeding.

All patients can be followed in this study for safety for at least 6 months after the start of treatment and 1 year for disease status.

Patients who discontinue study treatment for reasons other than disease progression (e.g. toxicity, withdrawal of consent) can continue to undergo scheduled tumor assessments every 90 days until the patient dies, experiences disease progression (intra- or extrahepatic), or initiates further systemic cancer therapy, whichever occurs first.

### Rescue Medication and Therapy

The study site will supply immunomodulatory rescue medication that will be obtained locally. The following rescue medications may be used:

### Medications

IL 6 receptor antibody Tocilizumab 4-8 mg/kg systemic IV over 60 minutes for CRS, may repeat as clinically indicated.
Methylprednisolone 2 mg/kg systemic IV bolus followed by 0.5 mg/kg IV every 6-12 hours for CRS Grade >2, or neurologic dysfunction. The first dose can be given without consultation with the investigator or designee; however, the subsequent doses must be given after the consultation with the study investigator or designee.
Consider 1 to 2 doses of anti-TNFα agents (infliximab or etanercept). The benefits are not known, but since TNFα may rise acutely, it is worth considering early in the disease process.
Etanercept 25 mg SC twice a week for 2 doses, given 3 to 4 days apart (0.4 mg/kg twice a week, maximum of 25 mg per dose.
Infliximab dose 10 mg/kg systemic IV weekly×2 doses.

### Interventions

Endoscopic cholangiography and stent placement.
Percutaneous cholangiography and stent placement.

Although the use of rescue medications is allowable at any time during the study, the use of rescue medications should be delayed, if possible and clinically appropriate, for at least 6 hours following the administration of study intervention. The date and time of rescue medication administration, as well as the name and dosage regimen of the rescue medication must be recorded.

Adverse events clearly due to disease progression, unrelated to study drug, or expected for the eligible patient population of the study are not considered DLTs.

### Assessment for Cytokine Release Syndrome

The Investigator, or designee, will assess each patient for the presence of CRS. CRS grading will be determined based on the NCI CTCAE v5.0 and managed as shown in Table 3.

TABLE 3
Grading Criteria for Cytokine Release Syndrome

| CRS | | |
|---|---|---|
| Severity | Manifestation | Management |
| Grade 1 | Fever (≥38° C.)^{a} with or without | Antipyretic and hypothermia blanket as needed |
| | constitutional symptoms, and/or | for fever |
| | ALT/AST >ULN-3 × ULN, | Assess for infection |
| | and/or | |
| | Alkaline phosphatase >ULN- | IV fluids as needed |
| | 2.5 × ULN, and/or | |
| | Bilirubin >ULN-1.5 × ULN | Symptomatic management of constitutional |
| | | symptoms and organ toxicities, as needed |
| Grade 2 | Hypotension (SBP <90 mmHg | Fever: Manage as per Grade 1 guidelines |
| | or <20% baseline) responding to | Hypotension: IV fluid bolus of 500-1000 mL |
| | fluids, and/or | NS, repeat as necessary to maintain SBP >90 |
| | Hypoxia responding to <40% | mmHg |
| | oxygen, and/or | If SBP refractory to NS boluses, consider the |
| | ALT/AST >3-5 × ULN, and/or | use of corticosteroids. |
| | Alkaline phosphatase >2.5- | If hypotension persists after IV fluid boluses and |
| | 5 × ULN, and/or | corticosteroids, consider: |
| | Bilirubin >1.5-3 × ULN | Transfer to ICU, vasopressin, 2D |
| | | echocardiogram |
| | | Hypoxia: Use supplemental oxygen as necessary |
| | | to keep oxygen saturation ≥92% |
| Grade 3 | Hypotension managed with one | Patient should be in the ICU: |
| | pressor, and/or | Fever: Manage as per Grade 1 CRS |
| | Hypoxia requiring ≥40% | Hypotension: |
| | oxygen, and/or | IV fluid boluses as needed as in Grade 2 CRS |
| | AST/ALT/Alkaline phosphatase | Vasopressors as needed |
| | >5-20 × ULN, and/or | Consider tocilizumab 8 mg/kg (maximum 800 |
| | Bilirubin >3-10 × ULN | mg per dose) IV for up to 3 doses in a 24-hour |
| | | period (maximum 4 doses total) |
| | | Obtain echocardiogram if not already performed |
| | | Hypoxia: |
| | | Use supplemental oxygen (≥6 L/min) including |
| | | high-flow oxygen delivery and non-invasive |
| | | positive pressure ventilation as needed |
| | | Use corticosteroids, IL-6 receptor antagonist as |
| | | above and supportive care |
| Grade 4 | Life-threatening consequences; | Fever: Manage as per Grade 1 CRS |
| | urgent intervention indicated, | Hypotension: |
| | and/or | IV fluids, IL-6 receptor antagonist, |
| | ALT/AST/Alkaline phosphatase | vasopressors, and hemodynamic monitoring as |
| | >20 × ULN, and/or | in Grade 3 |
| | Bilirubin >10 × ULN | High-dose corticosteroids |
| | | Hypoxia: |
| | | Positive pressure/mechanical ventilation |
| | | Use high-dose corticosteroids, IL-6 receptor |
| | | antagonist and supportive care |

Abbreviations:
2D = 2 dimensional;
ALT = alanine aminotransferase;
AST = aspartate aminotransferase;
ASTCT = American Society for Transplantation and Cellular Therapy;
CRS = cytokine release syndrome;
CTCAE = Common Terminology Criteria for Adverse Events;
ICU = intensive care unit;
IL = interleukin;
IV = intravenous;
NCI = National Cancer Institute;
NS = normal saline;
SBP = systolic blood pressure;
ULN = upper limit of normal
^{a}Fever is defined as temperature ≥38° C. not attributable to any other cause.
Source: Adapted from the NCI CTCAE and ASTCT (Lee DW, Santomasso BD, Locke FL, Ghobadi A, Turtle CJ, Brudno JN, et al. ASTCT consensus grading for cytokine release syndrome and neurologic toxicity associated with immune effector cells. Biol Blood Marrow Transplant. 2019; 25; 625-38).

### Imaging

Extent of disease will be measured radiographically at the time points for Cohort A, Cohort B, and Cohort C. Screening assessments must include an MRI of the abdomen and pelvis (with oral/IV Eovist contrast unless contraindicated) and a brain scan (CT with IV contrast or MRI). A spiral CT scan of the chest should be obtained in addition to the MRI of the abdomen and pelvis. If an MRI is medically contraindicated or at the discretion of the physician, a CT scan of the chest, abdomen, and pelvis may be performed using triple phase IV contrast. If a site is unable to perform a triple phase CT of the chest, abdomen, pelvis, they may perform the CT of the chest, abdomen, pelvis with IV contrast and obtain arterial phase and portal venous phase imaging. If a PET/CT scan is performed (not required), the CT portion of the study must be consistent with the standards for a full-contrast CT scan. Liver metastasis responses will be assessed on abdominal CT or MRI, while extrahepatic lesions will be assessed on whole body PET/CT scans or CT/MRI scans that cover the chest, abdomen, and pelvis. Hepatic imaging using MRI with Eovist contrast should be used whenever possible for assessment of liver tumors. The same imaging method used at screening must be used throughout the study.

Any evaluable or measurable disease must be documented at screening and re-assessed at each subsequent tumor evaluation. For patients with measurable disease, response will be assessed per RECIST v1.1. Local imaging reads will be utilized for response assessment during Phase 1b. Independent Central Review (ICR) for response assessment may be considered during Phase 2.

At the investigator's discretion, imaging may be performed at any time if PD is suspected. In addition, mRECIST and iRECIST assessments will be performed for secondary endpoint data collection but will not be incorporated into official response scoring.

### ECOG Performance Status

The ECOG PS scale will be used to assess how the disease is affecting the patient's daily living activities and ability to take care of themselves. At each specified time point, qualified site personnel will rate the patient according to the following scale:
Fully active, able to carry on all predisease performance without restriction
Restricted in physically strenuous activity but ambulatory and able to carry out work of a light or sedentary nature, e.g. light house work, office work
Ambulatory and capable of all self-care but unable to carry out any work activities; up and about more than 50% of waking hours
Capable of only limited self-care; confined to bed or chair more than 50% of waking hours
Completely disabled; cannot carry on any self-care; totally confined to bed or chair
Dead

Changes, i.e. worsening, will not be documented as AEs unless reported during the nondirected questioning for AEs.

### RECIST v1.1 Definitions

Measurable disease—The presence of at least 1 measurable lesion. If the measurable disease is restricted to a solitary lesion, its neoplastic nature should be confirmed by cytology/histology.

Measurable lesions—Lesion that can be accurately measured in at least 1 dimension with longest diameter ≥10 mm (CT scan slice thickness≤5 mm).

Nonmeasurable lesions—All other lesions, including small lesions (longest diameter <10 mm), as well as truly nonmeasurable lesions (such as leptomeningeal disease, ascites, pleural/pericardial effusion, inflammatory breast disease, lymphangitic involvement of skin or lung, abdominal masses that are not measurable by reproducible imaging techniques).

### Baseline Documentation

All measurable lesions up to a maximum of 2 lesions per organ and 5 lesions in total, representative of all involved organs should be identified as target lesions and recorded and measured at baseline.

Target lesions should be selected on the basis of their size (lesions with the longest diameter) and their suitability for accurate repeated measurements by consistent imaging techniques.

A sum of the longest diameter (LD) for all target lesions (non-nodal) will be calculated and reported as the baseline sum LD. The baseline sum LD will be used as reference by which to characterize the objective tumor response in the measurable dimension of the disease.

All other lesions (or sites of disease) should be identified as nontarget lesions and should also be recorded at baseline. Measurements of these lesions are not required, but the presence or absence of each should be noted throughout follow-up.

### Evaluation of Target Lesions

Complete Response (CR): Disappearance of all target lesions
Partial Response (PR): At least a 30% decrease in the sum of the LD of target lesions, taking as reference the baseline sum LD.
Progressive Disease (PD): At least a 20% increase in the sum of the LD of target lesions, taking as reference the smallest sum LD recorded since the treatment started or the appearance of 1 or more new lesions. The sum must demonstrate an absolute increase on 5 mm.
Stable Disease (SD): Neither sufficient shrinkage to qualify for PR nor sufficient increase to qualify for PD, taking as reference the smallest sum LD since the treatment started.

### Evaluation of Nontarget Lesions

Complete Response (CR): Disappearance of all nontarget lesions
Non-CR/Non-PD: Persistence of one or more nontarget lesion(s)
Progressive Disease (PD): Unequivocal progression of existing nontarget lesions, and/or the appearance of one or more new lesions.

### Assessment of Response

Response will be assessed at the time points for Cohort A, Cohort B, and Cohort C according to Table 4:

TABLE 4
Assessment of Response per RECIST v1.1
Time-Point Response

| | Nontarget | New | Overall |
|---|---|---|---|
| Target lesions | lesions | Lesions | response |
| CR | CR | No | CR |
| CR | Non-CR/non-PD | No | PR |
| PR | Non-PD | No | PR |
| SD | Non-PD | No | SD |
| PD | Any | Yes or No | PD |
| Any | PD | Yes or No | PD |
| Any | Any | Yes | PD |

Abbreviations: CR = complete response; PD = progressive disease; PR = partial response; SD = stable disease.

### Duration of Overall Response

The duration of overall response is measured from the time measurement criteria are met for CR or PR (whichever is first recorded) until the first date that recurrent or PD is objectively documented (taking as reference for PD the smallest measurements recorded since the treatment started). The duration of overall CR is measured from the time measurement criteria are first met for CR until the first date that PD is objectively documented. Duration of SD: Stable disease is measured from the start of the treatment until the criteria for progression are met, taking as reference the smallest sum of measurements recorded since the treatment started, including the baseline measurements.

### Overall Survival

For all patients, OS will be calculated from the date of enrollment to the time of death. Patients who are still alive prior to the data cutoff for final efficacy analysis, or who dropout prior to study end, will be censored at the day they were last known to be alive.

### Progression-Free Survival

For all patients, PFS will be calculated from the date of enrollment to the time of CT scan documenting relapse (or other unambiguous indicator of disease development), or date of death, whichever occurs first. Patients who have no documented relapse and are still alive prior to the data cutoff for final efficacy analysis, or who dropout prior to study end, will be censored at the date of the last radiological evidence documenting absence of relapse.

Modified RECIST (mRECIST)

mRECIST definitions for hepatocellular carcinoma are as follows:
Complete Response (CR)=Disappearance of any intratumoral arterial enhancement in all target lesions
Partial Response (PR)=At least a 30% decrease in the sum of diameters of viable (enhancement in the arterial phase) target lesions, taking as reference the baseline sum of the diameters of target lesions
Stable Disease (SD)=Any cases that do not qualify for either PR or progressive disease
Progressive Disease (PD)=An increase of at least 20% in the sum of the diameters of viable (enhancing) target lesions, taking as reference the smallest sum of the diameters of viable (enhancing) target lesions recorded since treatment started

Response will also be assessed by mRECIST as shown in Table 5.

TABLE 5
Assessment of Response per mRECIST

| | Nontarget | New | Overall |
|---|---|---|---|
| Target Lesions | Lesions | Lesions | Response |
| CR | CR | No | CR |
| CR | IR/SD | No | PR |
| PR | Non-PD | No | PR |
| SD | Non-PD | No | SD |
| PD | Any | Yes or No | PD |
| Any | PD | Yes or No | PD |
| Any | Any | Yes | PD |

Abbreviations: CR = complete response; PR = partial response; IR = incomplete response; SD = stable disease; PD = progressive disease.
Source: Lencioni R, Llovet JM. Modified RECIST (mRECIST) assessment for hepatocellular carcinoma. Sem Liver Dis. 2010; 30: 52-60.

RECIST 1.1 for Immune Based Therapeutics (iRECIST)

Response will also be assessed by iRECIST as shown in Table 6. In brief, the main differences between RECIST and iRECIST are explained in Seymour et al 2017 as follows: “The principles used to determine objective tumor response are largely unchanged from RECIST 1.1, but the major change for iRECIST is the concept of resetting the bar if RECIST 1.1 progression is followed at the next assessment by tumor shrinkage. iRECIST defines iUPD on the basis of RECIST 1.1 principles; however iUPD requires confirmation, which is done on the basis of observing either a further increase in size (or in the number of new lesions) in the lesion category in which progression was first identified in (i.e., target or nontarget disease), or progression (defined by RECIST 1.1) in lesion categories that had not previously met RECIST 1.1 progression criteria. However, if progression is not confirmed, but instead tumor shrinkage (compared with baseline), which meets the criteria of iCR, iPR or iSD, then the bar is reset so that iUPD needs to occur again (compared with nadir values) and then be confirmed (by further growth) at the next assessment for iCPD to be assigned. If no change in tumor size or extent from iUPD occurs, then the timepoint response would again be iUPD. This approach allows atypical responses, such as delayed responses that occur after pseudoprogression, to be identified, further understood, and better characterized.”

TABLE 6
Assessment of Response per iRECIST

| | | | Time-Point Response | |
|---|---|---|---|---|
| Target | Nontarget | New | No prior | |
| lesions* | lesions* | Lesions* | iUPD** | Prior iUPD**;*** |
| iCR | iCR | No | iCR | iCR |
| iCR | Non- | No | iPR | iPR |
| | iCR/non- | | | |
| | iUPD | | | |
| iPR | Non- | No | iPR | iPR |
| | iCR/non- | | | |
| | iUPD | | | |
| iSD | Non- | No | ISD | ISD |
| | iCR/non- | | | |
| | iUPD | | | |
| iUPD with | iUPD with | Yes | NA | NLs confirms iCPD if NLs were |
| no change | no change | | | previously identified and increase in |
| OR decrease | OR | | | size (≥5 mm in SOM for NLT or any |
| from last TP | decrease | | | increase for NLNT) or number. If no |
| | from last | | | change in NLs (size or number) from |
| | TP | | | last TP, remains iUPD |
| iSD, iPR, | iUPD | No | iUPD | Remains iUPD unless iCPD |
| iCR | | | | confirmed based on further increase |
| | | | | in size of NT disease (need not meet |
| | | | | RECIST 1.1 criteria for unequivocal |
| | | | | PD) |
| iUPD | Non- | No | iUPD | Remains iUPD unless iCPD |
| | iCR/non- | | | confirmed based on: |
| | iUPD | | | further increase in SOM of at least 5 |
| | | | | mm, otherwise remains iUPD |
| iUPD | iUPD | No | iUPD | Remains iUPD unless iCPD |
| | | | | confirmed based on further increase |
| | | | | in: |
| | | | | previously identified target lesion |
| | | | | iUPD SOM ≥5 mm or |
| | | | | NT lesion iUPD (prior assessment - |
| | | | | need not be unequivocal PD) |
| iUPD | iUPD | Yes | iUPD | Remains iUPD unless iCPD |
| | | | | confirmed based on further increase |
| | | | | in: |
| | | | | previously identified target lesion |
| | | | | iUPD ≥5 mm or |
| | | | | previously identified NT lesion iUPD |
| | | | | (need not be unequivocal) or |
| | | | | size or number of new lesions |
| | | | | previously identified |
| Non- | Non- | Yes | iUPD | Remains iUPD unless iCPD |
| iUPD/PD | iUPD/PD | | | confirmed based on: |
| | | | | increase in size or number of new |
| | | | | lesions previously identified |

Abbreviations:
CR = complete response;
iCPD = confirmed immune PD;
iCR = immune complete response;
iPR = immune partial response;
iRECIST = Response Evaluation Criteria in Solid Tumors for immune based therapeutics;
iSD = immune stable disease;
iUPD = immune unconfirmed PD;
NA = not applicable;
NL = new lesions;
NLT = new lesion target;
NLNT = new lesion nontarget;
NT = nontarget;
PD = progressive disease;
PR = partial response;
RECIST = Response Evaluation Criteria in Solid Tumors;
SD = stable disease;
SOM = sum of measures;
TP = time point
*Using RECIST 1.1 principles. If no pseudoprogression occurs, RECIST 1.1 and iRECIST categories for CR, PR and SD would be the same.
**in any lesion category.
***previously identified in assessment immediately prior to this TP.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A method for treating a primary liver cancer comprising administering to a subject in need thereof a therapeutically effective amount of a toll-like receptor 9 (TLR9) agonist having the structure: 5′-TCG AAC GTT CGA ACG TTC GAA CGT TCG AAT-3′ (SEQ ID NO: 1).

2. The method of claim 1, wherein the primary liver cancer is hepatocellular carcinoma (HCC) or intrahepatic cholangiocarcinoma (ICC).

3. The method of claim 1, wherein the TLR9 agonist is administered through a device by hepatic arterial infusion (HAI).

4. The method of claim 1, wherein the TLR9 agonist is administered through a device by portal vein infusion (PVI).

5. The method of claim 1, wherein the therapeutically effective amount of the TLR9 agonist administered is selected from the group consisting of 0.01 mg/mL, 0.04 mg/mL, 0.08 mg/mL, or 0.16 mg/mL.

6. The method of claim 1, wherein the therapeutically effective amount of the TLR9 agonist administered is selected from the group consisting of 0.5 mg, 2 mg, 4 mg, or 8 mg.

7. The method of claim 1, wherein the TLR9 agonist may be administered through a catheter device.

8. The method of claim 7, wherein the catheter device comprises a one-way valve that responds dynamically to local pressure and/or flow changes.

9. The method of claim 7, wherein the TLR9 agonist is administered through the catheter device via pressure-enabled drug delivery.

10. The method of claim 7, wherein the TLR9 agonist is administered for a period of time of about 10-200 minutes.

11. The method of claim 10, wherein the TLR9 agonist is administered for a period of time of about 10-60 minutes.

12. The method of claim 11, wherein the TLR9 agonist is administered for a period of time of about 25 minutes.

13. The method of claim 1, wherein the TLR9 agonist is administered in combination with one or more checkpoint inhibitors (CPIs), wherein the CPIs are administered systemically, either concurrently, before, or after the administration of the TLR9 agonist.

14. The method of claim 13, wherein the one or more CPIs include at least one of nivolumab, pembrolizumab, and ipilimumab.

15. The method of claim 1, wherein the administration of the TLR9 agonist comprises a dosing regimen comprising cycles, in which one or more of the cycles comprise the administration of the TLR9 agonist via a catheter device by hepatic arterial infusion followed by the systemic administration of the one or more checkpoint inhibitors (CPIs).

16. The method of claim 15, wherein one cycle comprises the administration of the therapeutically effective amount of TLR9 agonist once per week over three consecutive weeks.

17. The method of claim 16, wherein the dosing regimen comprises two cycles.

18. The method of claim 15, wherein the one or more CPIs include at least one of nivolumab, pembrolizumab, and ipilimumab.
